(19) United States
(12) Patent Application Publication
(10) Pub. No.: US 2022/0044328 A1
(43) Pub. Date: Feb. 10, 2022

(54) MACHINE LEARNING SYSTEMS AND METHODS TO EVALUATE A CLAIM SUBMISSION

(71) Applicant: Denialytics LLC, Duluth, GA (US)

(72) Inventor: Robert Ligon, Duluth, GA (US)

(21) Appl. No.: 17/235,245

(22) Filed: Apr. 20, 2021

Related U.S. Application Data
(63) Continuation-in-part of application No. 15/492,479, filed on Apr. 20, 2017, now abandoned.
(60) Provisional application No. 62/325,687, filed on Apr. 21, 2016.

(51) Int. Cl. G06Q 40/08; G06N 20/00; G06N 7/00; G16H 10/60

(52) U.S. Cl. CPC G06Q 40/08; G16H 10/60; G06N 7/00; G06N 20/00

(57) ABSTRACT

Embodiments of the present invention provide methods, apparatus, systems, computing devices, computing entities, and/or the like for evaluating an insurance claim. In accordance with one embodiment, a method is provided comprising: extracting data features for the insurance claim; processing the data features using a machine learning model to generate a plurality of potential denial data objects for a propensity to deny data object, wherein the propensity to deny data object represents a likelihood of the insurance claim being denied or underpaid and each potential denial data object represents a potential issue for the insurance claim and comprises a value representing an impact of the potential issue on the insurance claim being denied or underpaid; and processing at least one potential denial data object using a mitigating model to identify at least one mitigating action configured to cure the potential issue associated with the at least one potential denial data object.

### CROSS-REFERENCE TO RELATED APPLICATIONS

This application is a continuation-in-part of U.S. patent application Ser. No. 15/492,479, filed Apr. 20, 2017, entitled “Systems and Methods of Reducing Healthcare Claims Denials,” which claims the benefit of U.S. Provisional Patent Application Ser. No. 62/325,687, filed Apr. 21, 2016, the disclosures of which are hereby incorporated herein by reference in their entirety.

### TECHNICAL FIELD

The present disclosure is generally related to a computational framework making use of machine learning to evaluate the likelihood of the denial of a claim submission due to issues, defects, deficiencies, and/or the like found in the claim submission.

### BACKGROUND

Healthcare insurance claims may need certain information to be processed by an insurer. In some instances, insurance claims may be received by insurers without certain information, or with other deficiencies, and such insurance claims may be denied and/or underpaid by insurers. Insurance claims may be subject to coding standards that medical or administrative personnel are not familiar with, resulting in additional denied or underpaid claims due to incorrect coding. Oftentimes, denied or underpaid insurance claims are incorrectly denied or underpaid and are reversed after a healthcare provider or related entity appeals a denial or underpayment. For example, approximately 55-98% of denied claims may be overturned after appeal by a healthcare provider in certain instances.

### SUMMARY

In general, embodiments of the present invention provide methods, apparatus, systems, computing devices, computing entities, and/or the like for evaluating an insurance claim prior to submission. In accordance with one aspect, a method is provided. In particular embodiments, the method comprises: extracting, via one or more computer processors, a plurality of data features from data for the insurance claim; processing, via the one or more computer processors, one or more of the plurality of data features using a machine learning model to generate a plurality of potential denial data objects for an initial propensity to deny data object, wherein (i) the initial propensity to deny data object represents a likelihood of the insurance claim being at least one of denied or underpaid and (ii) each potential denial data object of the plurality of potential denial data objects represents a potential issue for the insurance claim and comprises a data value representing an impact of the potential issue on the insurance claim being at least one of denied or underpaid; processing, via the one or more computer processors, at least one potential denial data object of the plurality of potential denial data objects using a mitigating model to identify at least one mitigating action configured to cure the potential issue associated with the at least one potential denial data object; providing, via the one or more computer processors, the potential issue associated with the at least one potential denial data object, the data value corresponding to the at least one potential denial data object, and the at least one mitigating action for display via a user interface through a user device; receiving, via the one or more computer processors, an indication of a completion of the at least one mitigating action to cure the potential issue for the at least one potential denial data object; and responsive to receiving the indication of the completion of the at least one mitigating action: extracting, via the one or more computer processors, the plurality of data features from the data for the insurance claim, wherein the plurality of data features reflects the completion of the at least one mitigating action; processing, via the one or more computer processors, one or more of the plurality of data features using the machine learning model to generate the plurality of potential denial data objects for a working propensity to deny data object, wherein the working propensity to deny object represents a likelihood of the insurance claim being at least one of denied or underpaid in light of the completion of the at least one mitigating action and the at least one potential denial data object of the plurality of potential denial data objects comprises an updated data value based at least in part on the completion of the mitigating action; and providing, via the one or more computer processors, the potential issue associated with the at least one potential denial data object, the update data value corresponding to the at least one potential denial data object, and an indication that the at least one mitigating action has been completed for display via the user interface through the user device.

In accordance with another aspect, a system comprising one or more computer processors and computer memory storing computer-executable instructions is provided. In particular embodiments, the computer-executable instructions may be configured to, when executed by the one or more computer processors, cause the system to: extract a plurality of data features from data for the insurance claim; process one or more of the plurality of data features using a machine learning model to generate a plurality of potential denial data objects for an initial propensity to deny data object, wherein (i) the initial propensity to deny data object represents a likelihood of the insurance claim being at least one of denied or underpaid and (ii) each potential denial data object of the plurality of potential denial data objects represents a potential issue for the insurance claim and comprises a data value representing an impact of the potential issue on the insurance claim being at least one of denied or underpaid; process at least one potential denial data object of the plurality of potential denial data objects using a mitigating model to identify at least one mitigating action configured to cure the potential issue associated with the at least one potential denial data object; provide the potential issue associated with the at least one potential denial data object, the data value corresponding to the at least one potential denial data object, and the at least one mitigating action for display via a user interface through a user device; receive an indication of a completion of the at least one mitigating action to cure the potential issue for the at least one potential denial data object; and responsive to receiving the indication of the completion of the at least one mitigating action: extract the plurality of data features from the data for the insurance claim, wherein the plurality of data features reflects the completion of the at least one mitigating action; process one or more of the plurality of data features using the data feature machine learning model to generate the plurality of potential denial data objects for a working propensity to deny data object, wherein the working propensity to deny object represents a likelihood of the insurance claim being at least one of denied or underpaid in light of the completion of the at least one mitigating action and the at least one potential denial data object of the plurality of potential denial data objects comprises an updated data value based at least in part on the completion of the mitigating action; and provide the potential issue associated with the at least one potential denial data object, the update data value corresponding to the at least one potential denial data object, and an indication that the at least one mitigating action has been completed for display via the user interface through the user device.

In accordance with yet another aspect, a non-transitory computer-readable medium is provided. The non-transitory computer-readable medium may have computer-executable instructions stored therein, the computer-executable instructions configured to, when executed by one or more computer processors, cause the one or more computer processors to: extract a plurality of data features from data for the insurance claim; process one or more of the plurality of data features using a machine learning model to generate a plurality of potential denial data objects for an initial propensity to deny data object, wherein (i) the initial propensity to deny data object represents a likelihood of the insurance claim being at least one of denied or underpaid and (ii) each potential denial data object of the plurality of potential denial data objects represents a potential issue for the insurance claim and comprises a data value representing an impact of the potential issue on the insurance claim being at least one of denied or underpaid; process at least one potential denial data object of the plurality of potential denial data objects using a mitigating model to identify at least one mitigating action configured to cure the potential issue associated with the at least one potential denial data object; provide the potential issue associated with the at least one potential denial data object, the data value corresponding to the at least one potential denial data object, and the at least one mitigating action for display via a user interface through a user device; receive an indication of a completion of the at least one mitigating action to cure the potential issue for the at least one potential denial data object; and responsive to receiving the indication of the completion of the at least one mitigating action: extract the plurality of data features from the data for the insurance claim, wherein the plurality of data features reflects the completion of the at least one mitigating action; process one or more of the plurality of data features using the data feature machine learning model to generate the plurality of potential denial data objects for a working propensity to deny data object, wherein the working propensity to deny object represents a likelihood of the insurance claim being at least one of denied or underpaid in light of the completion of the at least one mitigating action and the at least one potential denial data object of the plurality of potential denial data objects comprises an updated data value based at least in part on the completion of the mitigating action; and provide the potential issue associated with the at least one potential denial data object, the update data value corresponding to the at least one potential denial data object, and an indication that the at least one mitigating action has been completed for display via the user interface through the user device.

In particular embodiments, a statistical model is used to process one or more of the plurality of data features for the insurance claim to generate one or more additional potential denial data objects for the initial propensity to deny data object. Here, each additional potential denial data object of the one or more additional potential denial data objects may represent an additional potential issue for the insurance claim and comprise a value representing an impact of the additional potential issue on the insurance claim being at least one of denied or underpaid. For instance, in some embodiments, the statistical model is configured to calculate the value for an additional potential denial data object of the one or more additional potential denial data objects as a quotient comprising a denied monetary amount for the additional potential denial data object divided by a total adjusted monetary amount over a historical period for a given payer.

In particular embodiments, a determination may be made as to whether the working propensity to deny data object satisfies a threshold. If so, in some embodiments, the insurance claim may be automatically submitted for payment. In addition, in particular embodiments, extracting the plurality of data features from the data for the insurance claim may involve transforming each of one or more of the plurality of data features comprising a categorical type of data feature into a numerical representation of the data feature, transforming each of one or more of the plurality of data features comprising a date type of data feature into a numerical representation of the data feature, and/or generating an additional data feature by performing a numerical operation on two of the one or more of the plurality of data features comprising the date type of data feature. In some embodiments, the machine learning model may comprise an ensemble of classifiers in which each classifier of the ensemble of classifiers is trained for providing a prediction for one of the plurality of potential denial data objects. Finally, in some embodiments, the user interface is embedded as one or more web service calls in at least one of a patient registration interface, a case management interface, or an electronic medical record user interface.

### BRIEF DESCRIPTION OF THE DRAWINGS

The detailed description is set forth with reference to the accompanying drawings. The drawings are provided for purposes of illustration only and merely depict example embodiments of the disclosure. The drawings are provided to facilitate understanding of the disclosure and shall not be deemed to limit the breadth, scope, or applicability of the disclosure. The use of the same reference numerals indicates similar, but not necessarily the same or identical components. Various embodiments may utilize elements or components other than those illustrated in the drawings, and some elements and/or components may not be present in various embodiments. The use of singular terminology to describe a component or element may, depending on the context, encompass a plural number of such components or elements and vice versa:

FIG. 1 is a flowchart of a process flow for training a machine learning model in accordance with various embodiments of the present disclosure;

FIG. 2 is an example of a machine learning model configuration in accordance with various embodiments of the present disclosure;

FIG. 3 is a flowchart of a process flow for extracting data features in accordance with various embodiments of the present disclosure;

FIG. 4 is a flowchart of a process flow for transforming categorical data features in accordance with various embodiments of the present disclosure;

FIG. 5 is a flowchart of a process flow for transforming a service code attribute data feature in accordance with various embodiments of the present disclosure;

FIG. 6 is a flowchart of a process flow for transforming date data features in accordance with various embodiments of the present disclosure;

FIG. 7 is a flowchart of a process flow for evaluating an insurance claim in accordance with various embodiments of the present disclosure;

FIG. 8 is a flowchart of a process flow for generating a propensity to deny data object in accordance with various embodiments of the present disclosure;

FIG. 9 is an example of a configuration used for generating a propensity to deny data object in accordance with various embodiments of the present disclosure;

FIG. 10 is another example of a configuration used for generating a propensity to deny data object in accordance with various embodiments of the present disclosure;

FIG. 11 is a flowchart of a process flow for generating one or more mitigating actions for one or more potential denial data objects in accordance with various embodiments of the present disclosure; and

FIG. 12 is a schematic block diagram of an illustrative server device in accordance with one or more example embodiments of the disclosure.

### DETAILED DESCRIPTION

Various embodiments for practicing the technologies disclosed herein are described more fully hereinafter with reference to the accompanying drawings, in which some, but not all embodiments of the technologies disclosed are shown. Indeed, the embodiments disclosed herein are provided so that this disclosure will satisfy applicable legal requirements and should not be construed as limiting or precluding other embodiments applying the teachings and concepts disclosed herein. Like numbers in the drawings refer to like elements throughout.

### Overview and Technical Contributions of Various Embodiments

This disclosure relates to, among other things, devices, systems, methods, computer-readable media, techniques, and methodologies for providing a computational framework to facilitate the evaluation of insurance claims to help in reducing denials of the claims. Certain embodiments of the framework are configured to generate a propensity to deny data object (also referred to as a propensity to deny score in some instances) for an insurance claim that represents a likelihood that the particular insurance claim may be denied or underpaid. Accordingly, in particular embodiments, the propensity to deny data object may be used to proactively address potential issues, defects, deficiencies, and/or the like that may be found in the insurance claim prior to submission, so as to increase a likelihood that the insurance claim will be approved and/or fully paid. For instance, in some embodiments, one or more mitigating actions may be recommended for one or more potential denial data objects represented within the propensity to deny data object to address potential issues, defects, deficiencies, and/or the like that may be found in the insurance claim. Here, the computational framework may identify one or more potential denial data objects for the insurance claim and one or more mitigating actions through the use of one or more machine learning models, statistical models, rule-based models, and/or clustering models. A potential denial data object may represent a specific potential issue, defect, deficiency, and/or the like that may be found in an insurance claim as identified by the computational framework. For example, a potential denial data object may represent a line item for the insurance claim that may contribute to a threat of the insurance claim being denied and/or underpaid. Accordingly, one or more mitigating actions may be performed to address (e.g., mitigate) the specific potential issue, defect, deficiency, and/or the like represented by the potential denial data object. In some embodiments, such mitigating actions may be automated so that the need for human intervention is not required to address the potential issue, defect, deficiency, and/or the like that may be found in the insurance claim.

Accordingly, embodiments of the disclosure can be configured to address potential issues, defects, deficiencies, and/or the like that may be found in insurance claims at the point of patient presentation, rather than forcing healthcare provider businesses to address claim issues, defects, deficiencies, and/or the like after a patient may no longer be available. For example, various embodiments of the computational framework allow for identifying and addressing conditions that can lead to a payer denying all or part of a provider's claims for reimbursement at the point of patient registration and during the patient's episode of care so that such conditions can be timely eliminated. As a result, embodiments of the computational framework increase the efficiency and utilization of provider and payer resources used in processing insurance claims, including computational, networking, and system resources.

As discussed further herein, various embodiments of the computational framework are configured to receive insurance claim data associated with an insurance claim. For example, the insurance claim data may include patient information, payer information, such as a payer identifier, claim code information, expected payment amount, billed amount, and/or the like. Here, embodiments of the framework are configured to generate a propensity to deny data object through the use of one or more machine learning models, as well as one or more statistical models in some embodiments. The propensity to deny data object represents a likelihood that the particular insurance claim will be denied and/or underpaid. Depending on the embodiment, the propensity to deny data object may include one or more suitable alphanumeric metrics, graphical indicators, or other indicia configured to indicate the likelihood the claim will be denied and/or underpaid. In particular embodiments, the propensity to deny data object may represent a plurality of potential denial data objects in which each potential denial data object may represent a potential issue, defect, deficiency, and/or the like that may be found in the insurance claim that could lead to a denial and/or underpayment of the claim. In some embodiments, a first propensity to deny data object may be generated as an initial propensity to deny data object, such as, for example, at a time of initial presentation. A second propensity to deny data object, or a working propensity to deny data object, may then be generated during a patient's stay as additional insurance claim data is collected or received and/or as one or more mitigating actions are performed to address the one or more potential issues, defects, deficiencies, and/or the like that may be found in the insurance claim to attempt to address the likelihood of the insurance claim being denied and/or underpaid.

Accordingly, in various embodiments, the computational framework is configured to make use of a mitigation model to generate the one or more mitigating actions that may be carried out to address the one or more potential issues, defects, deficiencies, and/or the like that may be found in the insurance claim. For example, in particular embodiments, the mitigating model may be a rules-based model configured to apply rules-based logic to identify one or more mitigating actions for various potential denial data objects representing the one or more potential issues, defects, deficiencies, and/or the like that may be found in the insurance claim. For example, if the insurance claim is coded incorrectly, resulting in a potential denial data object representing the code as an issue that can potentially lead to a denial of the claim, then the framework, via the mitigation model, may generate a mitigating action (e.g., a recommendation) for correcting the code.

In some embodiments, for example, the propensity to deny data object may be presented to a user through a user interface such as a graphical user interface, webpage, electronic report, electronic communication, and/or the like to alert the user to the potential that a claim may be rejected. In addition, the one or more mitigating actions may be communicated to the user through such an interface. Further, the user may be notified through the interface upon completion of a mitigating action (e.g., a stoplight changing from red to green upon completion of a mitigating action item, etc.).

As noted, in some instances, a mitigating action may be executed in an automated fashion without human intervention. For example, one or more systems may be used to automatically submit an authorization request to a payer needed for a medical service and/or treatment associated with an insurance claim, and/or to automatically retrieve missing data for an insurance claim from a data source. Furthermore, in particular embodiments, other actions may be carried out as a result of the propensity to deny data object meeting certain criteria and/or one or more of the mitigating actions being carried out, indicating a reduced likelihood of the insurance claim being denied and/or underpaid. For example, such other actions may involve submitting the insurance claim to the payer for payment. In some instances, such actions may be automated. Further details on various embodiments may be found in U.S. patent application Ser. No. 15/492,479, filed Apr. 20, 2017 and published as U.S. Patent Publication 2017/0308652, which claims the benefit of U.S. Provisional Patent Application No. 62/325,687, filed Apr. 21, 2016, in which the contents of both are hereby incorporated herein in their entirety.

Accordingly, various embodiments of the disclosure can be used in evaluating an insurance claim prior to submission of the insurance claim for payment. Such embodiments may enable the reduction of the number of healthcare insurance claims that are denied and/or underpaid by healthcare payers (e.g., insurance companies, Medicare, and/or the like). Further, embodiments of the disclosure may allow for healthcare service providers to address issues, defects, deficiencies, and/or the like found in insurance claims prior to submission for payment, and at a time when patients are available to assist in correcting such issues, defects, deficiencies, and/or the like. As a result, embodiments of the disclosure help facilitate better use of many healthcare service providers' resources, both personnel and systems resources, that are utilized in generating, managing, processing, and/or submitting insurance claims. Likewise, embodiments of the disclosure may facilitate better use of many healthcare payers' resources used in receiving, managing, and/or processing insurance claims.

In addition, various embodiments of the present disclosure provide a computational framework that can be used in supplementing, enhancing, replacing, and/or the like many existing processes used in generating, submitting, receiving, managing, and/or processing insurance claims that are oftentimes manually driven with a novel and improved automated process. As discussed further herein, various embodiments of the disclosure involve the use one or more machine learning models, statistical models, rule-based models, and/or clustering models in identifying and/or addressing potential issues, defects, deficiencies, and/or the like that may be found in insurance claims prior to their submission for payment. Accordingly, in particular embodiments, these various models are used in carrying out complex mathematical operations for identifying and/or addressing such potential issues, defects, deficiencies, and/or the like that may be found in insurance claims.

As a result, various embodiments of the present disclosure can carry out data processing that cannot be feasibly performed by a human, especially when such data processing involves the use of data from several different sources (e.g., patient registration, case management, electronical medical records, and/or the like). This is especially advantageous when the data processing must be carried out over a reasonable timeframe to allow for relevant observations to be gathered from the data and the insurance claims to be submitted in a timely and correct manner. Here, embodiments of the disclosure allow for the identification and correction of potential issues, defects, deficiencies, and/or the like that may be found in insurance claims prior to their submission for payment that would typically be infeasible to accomplish manually due to the vast complexity and sheer volume of information involved in generating, submitting, receiving, managing, and/or processing insurance claims. Thus, embodiments of the disclosure allow for data processing needed for identifying potential issues, defects, deficiencies, and/or the like that may be found in insurance claims, as well as identifying mitigating actions to address the potential issues, defects, deficiencies, and/or the like, to be carried out in a manner that is better suited for an automated setting. In addition, various embodiments of the present disclosure enhance the efficiency and speed of various computing systems, provide the ability to generate, manage, and/or process insurance claims that involve very large volumes of data, and make important contributions to various computational tasks that utilize real-time/expediated processing of such data. In doing so, various embodiments of the present disclosure make major technical contributions to improving the computational efficiency and reliability of various tasks involved in the generating, submitting, receiving, managing, and/or processing of insurance claims. This in turn translates to more computationally efficient software systems.

### Exemplary System Operation

The logical operations described herein may be implemented (1) as a sequence of computer implemented acts or one or more program modules running on a computing system and/or (2) as interconnected machine logic circuits or circuit modules within the computing system. The implementation is a matter of choice dependent on the performance and other requirements of the computing system. Accordingly, the logical operations described herein are referred to variously as states, operations, steps, structural devices, acts, or modules. These operations, steps, structural devices, acts, and modules may be implemented in software, in firmware, in special purpose digital logic, and any combination thereof. Greater or fewer operations may be performed than shown in the figures and described herein. These operations may also be performed in a different order than those described herein.

### Model Training Module

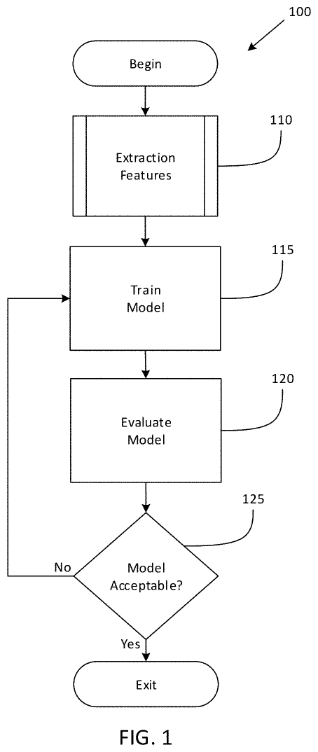

Turning now to FIG. 1, additional details are provided regarding a process flow for training a machine learning in accordance with various embodiments of the disclosure. Accordingly, FIG. 1 provides a flow diagram for a model training module for performing such functionality according to various embodiments of the disclosure.

As previously noted, various embodiments of the computational framework are configured for using a machine learning model in generating a propensity to deny (PTD) data object representing the likelihood of an insurance claim submitted by a healthcare provider being denied and/or underpaid by the corresponding healthcare payer. In particular embodiments, the machine learning model may be configured as a supervised or unsupervised model that is required to be trained. Accordingly, the machine learning model is trained so that the learning algorithm of the model can “learn” the relationship between the feature being predicted, in this instance the likelihood of the claim being denied and/or underpaid, and other features of the claim. Under supervised learning, the supervision refers to the fact that the target values found in training data provide a way for the model to know how well it has learned the desired task. In other words, given a set of training data, a supervised learning algorithm attempts to optimize the model to find the combination of features that result in the target output. In many instances, the training of the model continues until the model achieves a desired level of accuracy on the training data.

Accordingly, in various embodiments, the training data used in training the machine learning model may include, for example, ANSI 277, 835, and/or 837 Electronic Data Exchange (EDI) data sourced from one or more healthcare payers (e.g., insurance companies, Medicare, and/or the like). The 835 EDI transaction set is a set of source files that are healthcare claim payment and remittance advice that has been specified by the Health Insurance Portability and Accountability Act (HIPAA) 5010 requirements for the electronic transmission of healthcare payment and benefit information. An 835 EDI transaction is used primarily by healthcare insurance payers to make payments to healthcare providers, to provide Explanations of Benefits (EOBs), or both. When a healthcare service provider submits an 837 EDI transaction (healthcare claim), the insurance payer uses an 835 EDI transaction to detail the payment for that claim. The 837 EDI healthcare claim may include patient information such as: (1) a description of the patient; (2) the patient's condition for which treatment was provided; (3) the services provided; (4) the cost of the treatment; (5) and/or the like. While the 835 EDI transaction may include: (1) what charges were paid, reduced, or denied; (2) whether there was a deductible, co-insurance, co-pay, etc.; (3) any bundling or splitting of claims or line items; (4) how the payment was made, such as through a clearinghouse; (5) and/or the like. A particular 835 EDI transaction may not necessarily match up one-for-one with a specific 837 healthcare claim. In fact, it is not uncommon for multiple 835 EDI transactions to be used in response to a single 837 healthcare claim, or for one 835 EDI transaction to address multiple 837 healthcare claim submissions. As a result, 835 EDI transactions are important to healthcare providers, to track what payments were received for services they provided and billed.

The 277 EDI transaction set is a set of source files that are health care claim status responses used by healthcare payers to report on the status of 837 healthcare claims previously submitted by providers that has been specified by HIPAA for the submission of claim status information. A 277 EDI transaction can be used in one of the following three ways: (1) may be sent in response to a previously received 276 EDI Claim Status Inquiry; (2) may be sent by a payer to request additional information about a submitted claim (without a 276 EDI Claim Status inquiry); or (3) may be sent by a payer to provide claim status information to a provider using a 277 EDI transaction, without receiving a 276 EDI Claim Status inquiry. Information provided in a 277 EDI transaction generally indicates where the claim is in process, either as “pending” or “finalized.” If finalized, the 277 EDI transaction indicates the disposition of the claim, e.g., rejected, denied, approved for payment or paid. If the claim was approved or paid, payment information may also be provided in the 277 EDI transaction, such as method of payment, date, amount, and/or the like. If the claim has been denied or rejected, the 277 EDI transaction may include an explanation, such as if the patient is not eligible.

According, in various embodiments, an initial loading of historical data is performed that includes a certain amount (e.g., one year, etc.) of a provider's historical 277 EDI and 835 EDI transaction data. Additionally, in some embodiments, data feeds from the provider's payer contract data source may also be established as an additional source of historical data. In particular embodiments, the machine learning model is trained using supervised learning. Therefore, in these embodiments, the historical data is tagged with the corresponding output/correct answer (ground truth data). To develop the machine learning model, first the historical data is understood in order to determine which fields in the data to use as predictors and which ones to discard. For example, in particular embodiments, the following features found in the 837 EDI historical transactions may be identified to use as input for the machine learning model: a payer identification code; payer name; a plan type; a subscriber name suffix; a subscriber birth date; a subscriber gender; a patient name suffix; a patient birth date; a patient gender; a total charge amount; a claim onset symptoms date; a claim admission date; a claim discharge date; an admitting diagnosis; a service code identifier for a product or service; an attending provider last name; an attending provider first name; and/or the like.

In some embodiments, the historical 835 EDI transaction data may be used, while account payer contract terms enrichment is not used. Rather, payer contract terms may be used to enhance the accuracy of a propensity to deny prediction. The historical data may be partitioned in order to create a training data set and a scoring data set used to evaluate the machine learning model. For example, in particular embodiments, the historical data may be partitioned using an SPSS modeler.

As previously noted, the machine learning model is configured in various embodiments to generate a PTD data object for an insurance claim submission (e.g., an 837 EDI healthcare claim). Here, the PTD data object represents a likelihood of the insurance claim submission being denied and/or underpaid by the payer. In particular embodiments, the PTD data object may include one or more data objects, referred to herein as potential denial data objects, in which each data object may represent a particular potential issue, defect, deficiency, and/or the like that may be present (or not present) in the insurance claim submission that can lead to the claim being denied and/or underpaid. For example, in some embodiments, the one or more potential denial data objects may represent the adjustment reason codes provided in an 835 EDI transaction that are used by a payer to identify reasons for denying and/or underpaying on an insurance claim. In general, these codes describe why a claim was paid differently than it was billed. For example, the adjustment reason code PR32 indicates the payment on a particular claim was different than what was billed because the payer's records indicate the patient associated with the claim is not an eligible dependent. Accordingly, in other embodiments, the one or more potential denial data objects may represent other data that may be used in identifying issues, defects, deficiencies, and/or the like that may be found in insurance claims. However, for convenience, the one or more potential denial data objects are used to represent the different adjustment reason codes that may be provided in an 835 EDI transaction throughout the remainder of this disclosure. Although one of ordinary skill in the art should understand that the one or more potential denial data objects may represent other data in other embodiments and therefore use of the potential denial data objects to represent the different adjustment reason codes should not be viewed as limiting the scope of the disclosure.

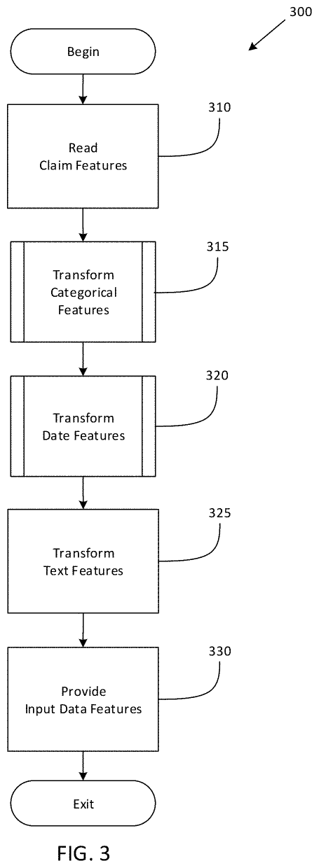

Accordingly, in various embodiments, the process flow 100 begins with the model training module extracting one or more data features for each insurance claim (e.g., data record thereof) found in the historical data in Operation 110. In particular embodiments, the model training module performs this particular operation by invoking a feature extraction module (FIG. 3) and the feature extraction module extracts the data features for each insurance claim found in the historical data. Depending on the embodiment, the data features may include different types of features such as quantitative features, categorical features, date features, text features, and/or the like. Therefore, as discussed further herein, the feature extraction module is configured in various embodiments to transform one or more features of each claim into numerical representations to prepare the features for use in machine learning.

In general, an insurance claim (837 EDI transaction) has a tree structure. Therefore, in particular embodiments, the model training module is configured to convert the data features for each of the insurance claims found in the historical data into a data structure once feature extraction has been completed. For example, the model training module may convert the data features for the insurance claims into a data structure such as a matrix in which each row of the matrix represents a claim found in the historical data and each column represent a different data feature of the claim.

In addition, in particular embodiments, the model training module may transform the targeted output for each of the insurance claims found in the historical data. For example, as discussed further herein, the targeted output for the machine learning model may be a data object having a feature structure representing different potential issues, defects, deficiencies, and/or the like that may be found in a claim that can lead to the claim being denied and/or underpaid. Here, the model training module may be configured to transform the targeted output for each insurance claim found in the historical data into a representation that can be used in machine learning such as, for example, the model training module may be configured in some embodiments to perform one-hot-encoding to transform the targeted output into a numerical representation.

At this point, the model training module trains the machine learning model using the extracted features for the insurance claims found in the training data set in Operation 115. A payer may deny paying a whole charged monetary amount value found in an insurance claim (837 EDI transaction), a portion of the charged monetary amount value for any service provided or deny payment on a claim level. Therefore, an 835 EDI transaction returned for a particular insurance claim may contain one or more adjustment reason codes and as a result, the machine learning model is configured in various embodiments as a multi-label classification model.

Accordingly, in these embodiments, the output of the model (e.g., the PTD data object) may be configured as a feature data structure having data features representing the different adjustment reason codes that may be provided in an 835 EDI transaction. For example, the PTD data object may be configured as a vector representation of a plurality of data features (potential denial data objects) in which each data feature (potential denial data object) found in the vector representation represents a different adjustment reason code. Here, in this example, a value may be provided for each potential denial data object that represents a likelihood of the adjustment reason code corresponding to the potential denial data object being provided in the 835 EDI transaction returned for the insurance claim. That is to say, a value may be provided for each potential denial data object that represents a likelihood of the insurance claim being denied and/or underpaid based at least in part on the issue, defect, deficiency, and/or the like associated with the adjustment reason code corresponding to the potential denial data object.

Accordingly, in particular embodiments, the machine learning model is arranged using a one-vs-the-rest (OvR) configuration to predict multiple labels for a particular insurance claim. For instance, in some embodiments, one model (e.g., classifier) is fitted per class (e.g., potential denial data object), and for each model, the class is fitted against all the other classes. Here, for example, the models may be based at least in part on Gradient Boosted Decision Trees (GBDT). Gradient boosting is a machine learning technique for regression and classification problems that produces a prediction model in the form of an ensemble of weak predictions models, typically decision trees. The goal of the ensemble is to combine the predictions of several base estimators built with a given learning algorithm to improve generalizability and/or robustness over a single estimator.

For example, these models can be built sequentially with the first model learning how to fit to the target variable, the second model learning how to fit to the residual (difference) between the predictions of the first model and the ground truth, the third model learning how to fit the residuals between the second model and the ground truth, and so on. All the models can be trained by propagating the gradients of errors throughout the entire ensemble. Accordingly, GBDT is a generalization of boosting to arbitrary differentiable loss functions. Thus, in boosting, base estimators can be built sequentially with the goal of reducing the bias of the combined model.

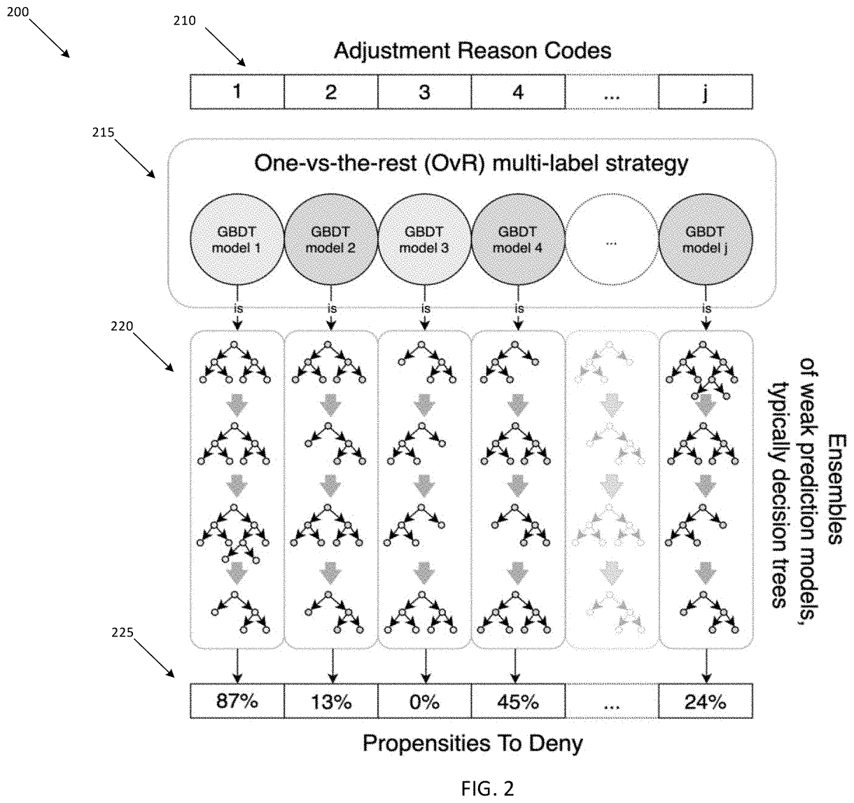

Turning briefly to FIG. 2, an example of a machine learning model configuration 200 is provided that can be used in accordance with various embodiments. Here, the configuration 200 is based at least in part on the OvR multi-label strategy with each potential denial data object (e.g., adjustment reason code 210) having a model 215 configured as an ensemble of prediction models 220 to produce a corresponding value 225 for the potential denial data object. In turn, the value 225 for each potential denial data object provides a prediction (likelihood) of the corresponding adjustment reason code 210 being returned in the 835 EDI transaction for the insurance claim. That is to say, the value 225 provides a prediction of the insurance claim being denied and/or underpaid based at least in part on the issue, defect, deficiency, and/or the like associated with the corresponding adjustment reason code 210 being found in the insurance claim.

Returning to FIG. 1, the model training module continues with evaluating the trained machine learning model in Operation 120. Here, the model training module makes use of the scoring data set (testing data set) as previously discussed. In general, the purpose of evaluating the machine learning model in various embodiments is to test and validate the model's efficiency. As previously noted, the historical data is divided into training and scoring data sets. For example, in particular embodiments, a software tool may be used during training to provide an implementation of iterative stratification which aims to provided well-balanced distribution of evidence of label relations up to a given order.

Depending on the embodiment, metrics such as precision, recall, F1 score, micro average, macro average, weighted average, sample average, and/or the like are used to evaluate a model (e.g., classifier). Precision is the ability of the model (e.g., classifier) not to label an instance positive if it is negative. Accordingly, in particular embodiments, precision is defined for each class as the ratio of true positives to the sum of true and false positives (e.g., precision−accuracy of positive predictions, precision=true positives/(true positives+false positives)). Recall is the ability of a model (e.g., classifier) to find all positive instances. In particular embodiments, recall is defined for each class as the ratio of true positives to the sum of true positives and false positives (recall−fraction of positives that were correctly identified, recall=true positives/(true positives+false negatives). F1 score for a class is a weighted harmonic mean of precision and recall such that the best score is 1.0 and the worst is 0.0. F1 scores are lower than accuracy measures as they embed precision and recall into their computation. Accordingly, the weighted average of F1 is often used to compare classifier models, not global accuracy (F1 score=2*(recall*precision)/(recall+precision). Micro average is averaging the total true positives, false negatives, and false positives. Macro average is averaging the unweighted mean per label. Weighted average is averaging the support-weighted mean per label.

Thus, in various embodiments, the model training module is configured to evaluate the efficiency of the machine learning model using one or more of the metrics described above. Here, in particular embodiments, the model training module may be configured to determine whether the machine learning model performs to an acceptable level (e.g., threshold level) of efficiency in Operation 125. In some embodiments, the model training module may be configured to compare the quality of the model with a baseline model that is a dummy estimator that generates random predictions within the training set class distribution.

Therefore, if the model training module determines the efficiency of the machine learning model is not acceptable, then the model training module returns to Operation 115 and further trains the model. Although not shown in FIG. 1, the further training may entail gathering further historical data that can be used in training the machine learning model and/or re-partitioning the historical data into the training and scoring data sets. If the model training module determines the efficiency of the machine learning model is acceptable, then the process flow 100 is ended. At this point, the machine learning model may then be used in evaluating insurance claims for identifying potential issues, defects, deficiencies, and/or the like that may be found in the insurance claims that may result in the insurance claims being denied and/or underpaid. Further detail is now provided on the use of the machine learning model.

Further, it is noted that in various embodiments active learning may be used to improve the efficiency of the machine learning model as data is collected on PTD data objects generated by the machine learning model for various insurance claims and the actual outcome of the insurance claims once submitted to payers. For instance, in particular embodiments, the model training module may be invoked after the initial training of the machine learning model to further train the model using the data collected on the PTD data objects generated by the machine learning model for the insurance claims and the actual outcome of the insurance claims. For example, the model training module may be configured to run as a batch process (e.g., nightly) on a current day's 835 EDI transactions and corresponding 837 EDI transactions to further “fine-tune” the machine learning model in a closed-loop fashion to improve the efficiency of the model. Accordingly, such a configuration may support a comparison of the PTD data objects generated by the machine learning model for insurance claims with the actual outcomes for the claims. For example, if the machine learning model generated a PTD data object for a particular insurance claim indicating a very low likelihood of the claim being denied but regardless the claim was actually denied, then the model training module may use this outcome to further train the machine learning model to learn from this discrepancy and recalibrate accordingly. As noted, this may allow for the machine learning model to improve its accuracy.

### Feature Extraction Module

Turning now to FIG. 3, additional details are provided regarding a process flow for extracting data features from the data for an insurance claim in accordance with various embodiments of the disclosure. Accordingly, FIG. 3 provides a flow diagram showing a feature extraction module for performing such functionality according to various embodiments of the disclosure.

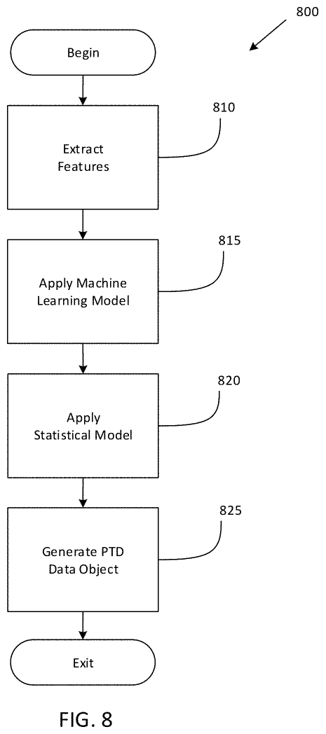

As previously noted, the feature extraction module is invoked in various embodiments by the model training module to extract data features from historical data as described above. However, with that said, the feature extraction module may be invoked by a different module or as a stand-alone module in other embodiments. For example, as discussed further herein, the feature extraction module is invoked in various embodiments by a generate propensity to deny data object module (FIG. 8) to extract the data features from the data for an insurance claim that is being evaluated.

The process flow 300 begins with the feature extraction module reading the data features from data for the insurance claim in Operation 310. As previously noted, the data features may include different types of data features such as, for example, quantitative features, categorical features, date features, text features, and/or the like. Therefore, in particular embodiments, the feature extraction module is configured to transform one or more data features for various types of data features into a more suitable representation that can be used for machine learning.

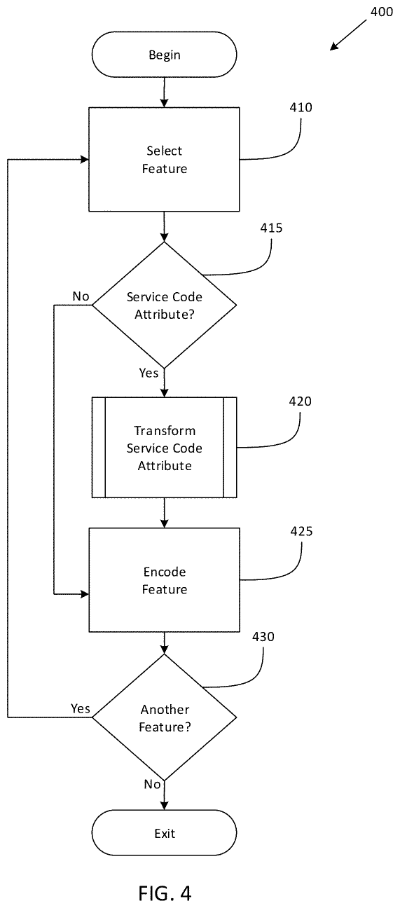

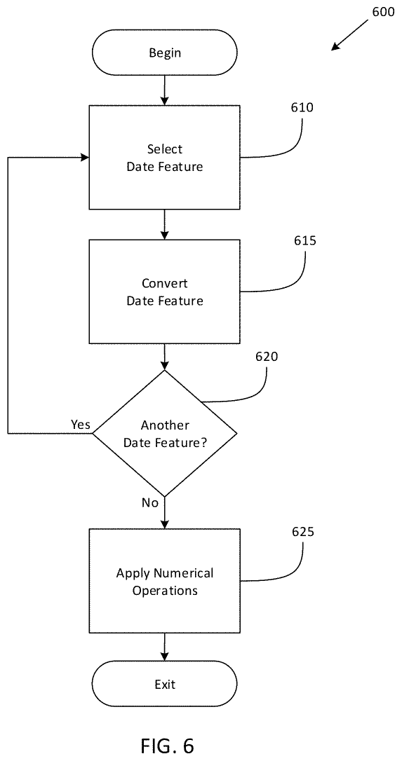

Accordingly, in various embodiments, the feature extraction module transforms the data features involving categorical data in Operation 315. In particular embodiments, the feature extraction module performs this particular operation by invoking a transform categorical feature module (FIG. 4) and in turn, the transform categorical feature module transforms each of the categorical data features (data thereof) into a numerical representation. Likewise, in various embodiments, the feature extraction module transforms the data features involving date data in Operation 320. In particular embodiments, the feature extraction module performs this particular operation by invoking a transform date feature module (FIG. 6) and in turn, the transform date feature module transforms each of the date data features (data thereof) into a numerical representation. In addition, in some embodiments, the transform date feature module may generate additional data features by applying numerical operations to one or more of the transformed date data features.

Continuing, in various embodiments, the feature extraction module transforms the data features involving text data in Operation 325. Here, in particular embodiments, the feature extraction module may be configured to transform each of the text data features by using one or more natural language processing techniques such as, for example, a word embedding technique to transform a text data feature into an embedded feature representation of the text data feature. Accordingly, although not shown in FIG. 3, the feature extraction module may be configured in other embodiments to transform other types of data features as those of ordinary skill in the art can envision in light of this disclosure. Once the feature extraction module has completed extracting and transforming all of the data features from the data for the insurance claim, the feature extraction module returns the data features in Operation 330 and the process flow 300 ends.

### Transform Categorical Feature Module

Turning now to FIG. 4, additional details are provided regarding a process flow for transforming categorical data features in accordance with various embodiments of the disclosure. Accordingly, FIG. 4 provides a flow diagram showing a transform categorical feature module for performing such functionality according to various embodiments of the disclosure.

As previously noted, the transform categorical feature module is invoked in various embodiments by the feature extraction module to transform categorical data features for an insurance claim as described above. However, with that said, the transform categorical feature module may be invoked by a different module or as a stand-alone module in other embodiments.

The process flow 400 begins with the transform categorical feature module selecting a categorical data feature for the insurance claim in Operation 410. Once selected, the transform categorical feature module determines whether the selected feature is for a service code attribute in Operation 415. A service code attribute is a categorical data feature that is provided as an identifier for one or more products and/or services rendered by the provider submitting the insurance claim. Here, in particular embodiments, the service code attribute may involve a list of service codes and may be found in the “Product/ServiceID” value in the “Professional Service (SV1)” segment or the “Institutional Service Line (SV2)” segment in the Loop 2400 of an insurance claim submission (an 837 EDI transaction).

Thus, if the transform categorical feature module determines the selected categorical data feature is the service code attribute, then the transform categorical feature module transforms the service code attribute in Operation 420. Accordingly, in various embodiments, the transform categorical feature module performs this particular operation by invoking a transform service code attribute module. The number, type, and order of the service codes provided in the service code attribute can vary from claim to claim. Therefore, in particular embodiments, the transform service code attribute module is configured to transform the list of service codes found in the service code attribute into a data representation (e.g., vector) in N dimensional space.

If the transform categorical feature module determines the selected categorical data feature is not the service code attribute, then the transform categorical feature module encodes the selected categorical data feature in Operation 425. Here, in various embodiments, the transform categorical feature module is configured to encode the selected categorical data feature into a numerical representation by replacing the categorical data for the feature by a numeric. For example, the selected categorical data feature may be plan type, which is a code identifying the type of claim. Here, the transform categorical feature module may assign a particular number (e.g., 1, 2, 3, etc.) to the data feature based at least in part on the plan type represented by the categorical data found in this data feature.

Once the transform categorical feature module has transformed and/or encoded the selected categorical data feature, the transform categorical feature module determines whether another categorical data feature is provided for the insurance claim in Operation 430. If so, then the transform categorical feature module returns to Operation 410, selects the next categorical data feature for the insurance claim, and processes the data feature as just described above. Once the transform categorical feature module has processed all of the categorical data features for the insurance claim, the process flow 400 ends.

### Transform Service Code Attribute Module

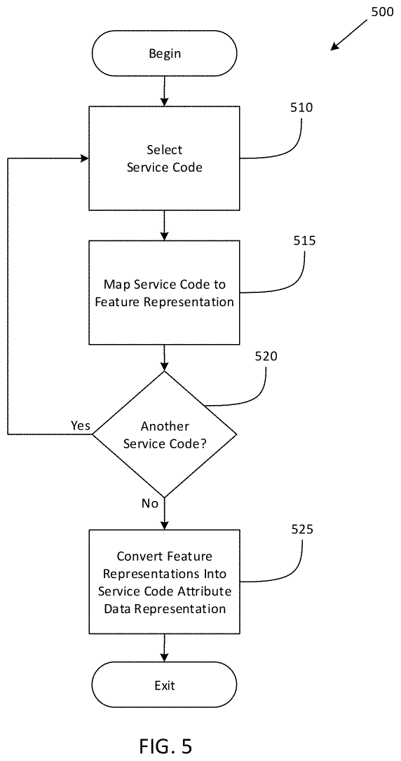

Turning now to FIG. 5, additional details are provided regarding a process flow for transforming the service code attribute for an insurance claim in accordance with various embodiments of the disclosure. Accordingly, FIG. 5 provides a flow diagram showing a transform service code attribute module for performing such functionality according to various embodiments of the disclosure.

As previously noted, the transform service code attribute module is invoked in various embodiments by the transform categorical feature module to transform the list of service codes found in the service code attribute for an insurance claim into a data representation as described above. However, with that said, the transform service code attribute module may be invoked by a different module or as a stand-alone module in other embodiments.

Accordingly, in various embodiments, a combination of term frequency-inverse document frequency (TF-IDF) vectorization and a Word2Vec technique are used in creating an embedded feature representation of the service code attribute data feature. Therefore, the process flow 500 begins with the transform service code attribute module selecting a service code found in the service code attribute data feature for the insurance claim in Operation 510.

Once selected, the transform service code attribute module maps the selected service code to a feature representation in Operation 515. Here, in particular embodiments, each service code that may be found in the service code attribute data feature for an insurance claim may be transformed into a feature representation using one or more Word2Vec techniques. An underlying assumption of Word2Vec is that two service codes sharing similar contexts are assumed to share a similar embedded feature representation. Therefore, a dictionary may be created using a Word2Vec technique that maps each service code into a N-dimensional embedded feature representation (e.g., a 100-dimensional vector representation) and the transform service attribute module is configured to identify the N-dimensional embedded feature representation from the dictionary for the selected service code.

The transform service code attribute module determines whether another service code in present in the service code attribute data feature in Operation 520. If so, then the transform service code attribute module returns to Operation 510, selects the next service code from the service code attribute data feature, and maps the newly selected service code to its corresponding N-dimensional embedded feature representation in Operation 515.

Once the transform service code attribute module has mapped each of the service codes found in the service code attribute data feature to a corresponding N-dimensional embedded feature representation, the transform service code attribute module converts the N-dimensional embedded feature representations for the service codes into a service code attribute feature representation in Operation 525. For example, in particular embodiments, the transform service code attribute module may be configured to perform the conversion by averaging the N-dimensional embedded feature representations for all the service codes. In some embodiments, the elements of the data representation may be further multiplied by corresponding coefficients that are pre-trained using a TF-IDF vectorizer. As result, the transform service code attribute module in various embodiments provides a N-dimensional feature representation (e.g., 100-dimensional vector representation) of the service codes found in the service code attribute data feature for the insurance claim.

### Transform Date Feature Module

Turning now to FIG. 6, additional details are provided regarding a process flow for transforming date data features in accordance with various embodiments of the disclosure. Accordingly, FIG. 6 provides a flow diagram showing a transform date feature module for performing such functionality according to various embodiments of the disclosure.

As previously noted, the transform date feature module is invoked in various embodiments by the feature extraction module to transform date data features for an insurance claim as described above. However, with that said, the transform date feature module may be invoked by a different module or as a stand-alone module in other embodiments.

The process flow 600 begins with the transform date feature module selecting a date data feature for the insurance claim in Operation 610. Once selected, the transform date feature module converts the selected date data feature into a numerical representation in Operation 615. Accordingly, in various embodiments, a date data feature may be transformed into a quantitative and/or categorical feature such as, for example, year, month, day, day of week, day of the year, and/or the like. Once transformed, the transform data feature module determines whether another date data feature is present for the insurance claim in Operation 620. If so, then the transform date feature module returns to Operation 610, selects the next date data feature, and transforms the newly selected date data feature in Operation 615.

In particular embodiments, the transform date feature module is configured to generate one or more data features by applying numerical operations (e.g., addition, subtraction, multiplication, division, and/or the like) to one or more date data features. Therefore, in these embodiments, the transform date feature module applies numerical operations to the one or more date data features accordingly in Operation 625. For example, the transform data feature module may generate a data feature by subtracting a patient's birth date from an admission date to get a data feature representing the age of the patient on the day he or she was admitted to the healthcare provider (e.g., hospital). Accordingly, such additional data features may be beneficial in improving the machine learning model's efficiency in that these additional data features may reveal additional hidden relations in the data.

### Evaluate Claim Module

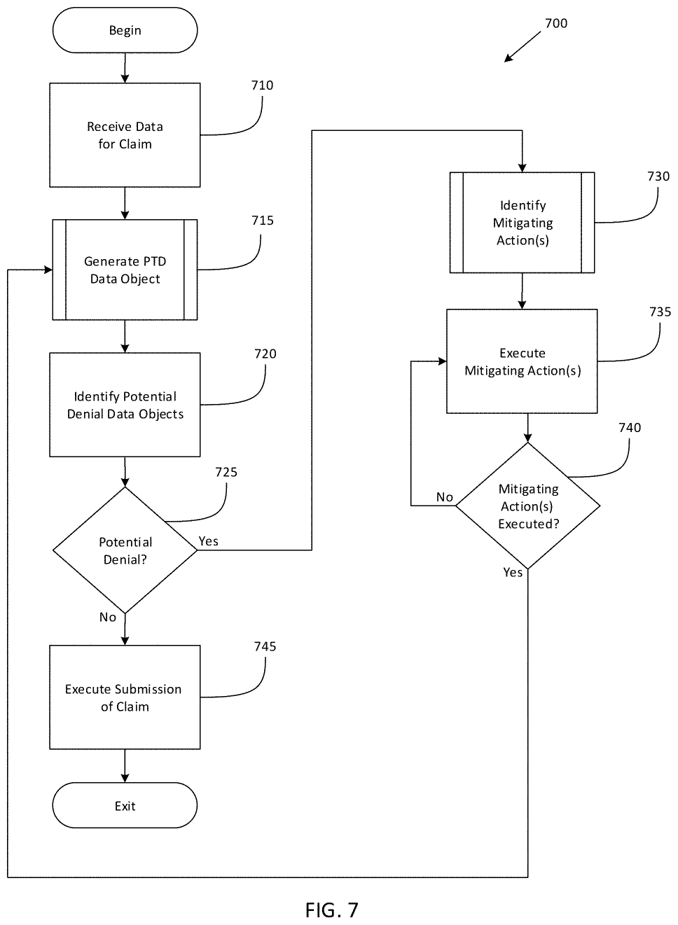

Turning now to FIG. 7, additional details are provided regarding a process flow for evaluating an insurance claim in accordance with various embodiments of the disclosure. Accordingly, FIG. 7 provides a flow diagram for an evaluate claim module for performing such functionality according to various embodiments of the disclosure.

As previously noted, various embodiments of the computational framework are configured for using a machine learning model in generating a PTD data object representing the likelihood of an insurance claim submitted by a healthcare provider being denied and/or underpaid by the corresponding healthcare payer. Accordingly, in various embodiments, the computational framework can be used in guiding the healthcare provider through a cognizant real-time process to facilitate having insurance claims submitted by the healthcare provider to a healthcare payer from being denied and/or underpaid. Here, embodiments of the framework may provide the healthcare provider with forewarning on potential issues, defects, deficiencies, and/or the like that may lead to an insurance claim being denied and/or underpaid. In addition, embodiments of the framework may provide one or more mitigating actions that may be carried out to cure and/or remedy the potential issues, defects, deficiencies, and/or like. With this in mind, the computational framework in various embodiments may include an evaluate claim module configured for carrying out functionality for evaluating insurance claims to identify potential issues, defects, deficiencies, and/or the like that may be found in the claims and providing mitigating actions to cure and/or remedy the potential issues, defects, deficiencies, and/or the like.

Therefore, the process flow 700 begins in various embodiments with the evaluate claim module receiving the data for an insurance claim in Operation 710. Depending on the embodiment, the evaluate claim module may receive the data for the claim from different data instruments and/or different data sources. For example, as previously discussed, the data for an insurance claim may be provided via an 837 EDI transaction. Here, for example, the evaluate claim module may “receive” the data for the insurance claim by being provided the data though some type of interface such as a user interface in which a user enters (e.g., uploads) the data for the insurance claim such as the 837 EDI transaction that is (or has been) submitted to a payer to initiate payment on the claim. While in other instances, the evaluate claim module may be configured to “receive” the data for the insurance claim through some type of interface such as an application programming interface (API) to one or more systems for the healthcare provider. For example, in particular embodiments, the data for the insurance claim may be provided as one or more admitted, discharged, transferred (ADT) messages that are used within a healthcare provider to communicate events about a patient to different clinical systems for the healthcare provider. Accordingly, depending on the embodiment, the evaluate claim module may “receive” the data for the insurance claim by being provided the data from some entity (e.g., user, system, data source, and/or the like) or by accessing the data from some entity.

Once the data for the insurance claim has been received, the evaluate claim module generates a PTD data object for the insurance claim in Operation 715. Here, in particular embodiments, the evaluate claim module is configured to perform this operation by invoking a generate propensity to deny data object module (FIG. 8) and in turn, the generate propensity to deny data object module generates a PTD data object for the insurance claim.

As previously discussed, the PTD data object in various embodiments represents a likelihood of the insurance claim being denied and/or underpaid by the payer. At noted, in particular embodiments, the PTD data object may include one or more potential denial data objects in which each potential denial data object may represent a particular issue, defect, deficiency, and/or the like that may be present (or not present) in the insurance claim that can lead to the claim being denied and/or underpaid. For example, in some embodiments, the one or more potential denial data objects represent the adjustment reason codes provided in an 835 EDI transaction that are used by a payer to identify reasons for denying and/or underpaying on an insurance claim.

Accordingly, each potential denial data object found in the PTD data object provides a representation (e.g., value) of a likelihood the corresponding issue, defect, deficiency, and/or the like exists for the insurance claim that may lead to the insurance claim being denied and/or underpaid. Therefore, in particular embodiments, the evaluate claim module identifies the potential denial data objects from the PTD data object that may lead to the insurance claim being denied and/or underpaid in Operation 720. For instance, in some embodiments, the evaluate claim module may identify those potential denial data objects from the PTD data object having a value satisfying a threshold value (e.g., having a value meeting or exceeding a threshold value).

At this point, the evaluate claim module determines whether any potential denial data objects have been identified for the insurance claim in Operation 725. If so, then the evaluate claim module generates one or more mitigating actions for the identified potential denial data objects in Operation 730. As previously discussed, a mitigating action may be carried out to cure and/or remedy a potential issue, defect, deficiency, and/or the like associated with an identified potential denial data object. For example, the identified potential denial data object may represent an adjustment reason code associated with denying an insurance claim because a service identified in the claim for reimbursement requires a pre-authorization by the payer that has not been acquired by the healthcare provider. Therefore, in this example, a mitigating action may be to have the healthcare provider acquire the pre-authorization from the payer.

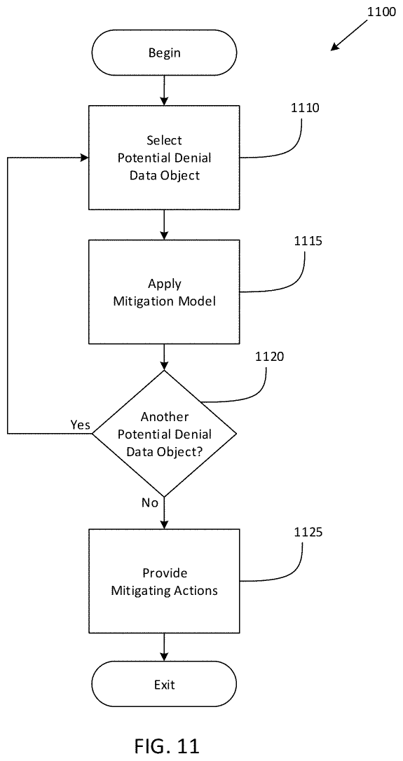

Accordingly, in particular embodiments, the evaluate claim module generates the one or more mitigating actions for the identified potential denial data objects by invoking a mitigating action module (FIG. 11). As discussed further herein, the mitigating action module is configured to process each of the potential denial data objects using a mitigating action model to identify one or more mitigating actions that can be performed to cure and/or remedy the potential issue, defect, deficiency, and/or the like associated with the potential denial data object.

Once the one or more mitigating actions have been generated for the identified potential denial data objects, the evaluate claim module executes the mitigating action(s) in Operation 735. Accordingly, depending on the embodiment, executing the mitigating actions may entail different operations, mechanisms, user interfaces, APIs, and/or the like. For instance, in particular embodiments, the evaluate claim module may be configured to provide the PTD data object, denial data object(s), and/or mitigating action(s) for display on one or more user interfaces. For example, using web service calls, embodiments of the evaluate claim module can be embedded into patient registration, case management and electronic medical record user interfaces. Specifically, for example, the user may gain access to the evaluate claim module via a website and the evaluate claim module may provide the PTD data object, denial data object(s), and/or mitigating action(s) to display on one or more webpages of the website through a browser executing on a computing entity being used by the user. Here, the user may be directed to take one or more mitigating actions to eliminate each of the denial data objects (that is, to cure and/or remedy the potential issue, defect, deficiency, and/or the like associated with the denial data object).

Accordingly, the evaluate claim module (or some other module) may be configured in some embodiments to keep track of the user's actions to cure and/or remedy potential issues, defects, deficiencies, and/or the like identified for the insurance claim. For example, a “stop light” graphical indicator may be displayed on the user interface that changes a denial data object (e.g., the corresponding potential issue, defect, deficiency, and/or the like) from red to green once the user has completed one or more mitigating actions to cure and/or remedy the corresponding potential issue, defect, deficiency, and/or the like.

While in other embodiments, executing the mitigating action(s) may entail performing one or more automated operations to cure and/or remedy a potential issue, defect, deficiency, and/or the like identified for the insurance claim. For instance, the evaluate claim module may automatically populate an electronic form and submit the form via an API to a corresponding system. For example, the evaluate claim module may populate an electronic form requesting pre-authorization for a healthcare service from a payer and submit the form via an API to a corresponding system of the payer to request the pre-authorization.

In other example, the evaluate claim module may submit a request for a medical service and/or diagnostic test (e.g., x-ray) for a patient to address data associated with the service or test required on the insurance claim. Again, the evaluate claim module may be configured to submit the request to one or more of the healthcare provider's systems used in scheduling such service and/or test for the patient. Yet, in another example, the evaluate claim module may access and retrieve additional data from some external data source to gather further data needed on the insurance claim to address the potential issue, defect, deficiency, and/or the like identified for the insurance claim. In other examples, the evaluate claim module may execute mitigation action(s) involving processes using autodialing, payer portal screen scraping and natural language technology, thus reducing manual intervention and account “touches.” Those of ordinary skill in art can envision other operations, mechanisms, user interfaces, APIs, and/or the like that may be used in executing the mitigating action(s) in light of this disclosure.

Accordingly, in particular embodiments, the evaluate claim module may be configured to monitor and determine when one or more of the mitigating actions have been performed in Operation 740. If the evaluate claim module determines one or more mitigating actions have been performed, then the evaluate claim module returns to Operation 715 in some embodiments and generates an updated PTD data object for the insurance claim. As a result, the evaluate claim module may then further evaluate the insurance claim with respect to what affect the completed mitigating action(s) had on the likelihood of the insurance claim being denied and/or underpaid.

For instance, in some embodiments, the evaluate claim module may generate a first PTD data object as an initial PTD data object, such as, for example, at a time of initial presentation. Accordingly, the evaluate claim module then then generate one or more working PTD data objects during a patient's stay as additional insurance claim data is collected or received and/or as one or more mitigating actions are performed to address the one or more potential issues, defects, deficiencies, and/or the like with respect to the insurance claim to attempt to address the likelihood of the insurance claim being denied and/or underpaid.

In particular embodiments, the evaluate claim module may be configured to execute one or more other actions (e.g., actions other than mitigating actions), such as submit the insurance claim in Operation 745, in response to the evaluate claim module not identifying any further potential denial data objects that are problematic for the insurance claim. For example, the evaluate claim module may engage an automated payer request/response system, payer portal, and/or provider services. At that point, the evaluate claim module may discontinue evaluating the insurance claim and the process flow 700 may end.

Therefore, as a result of using the evaluate claim module to evaluate insurance claims to identify any potential issues, defects, deficiencies, and/or the like for the insurance claims that may cause the claim to be denied and/or underpaid, as well as identify and execute mitigating action(s) to address the potential issues, defects, deficiencies, and/or the like prior to submitting the insurance claims to payers for payment, the evaluate claim module can enable healthcare providers in minimizing having insurance claims denied and/or underpaid by healthcare payers. Accordingly, such use of the evaluate claim module may provide a healthcare provider with improved efficiency and use of limited resources, such as personnel and processing systems, used in generating, managing, submitting, and/or the like insurance claims as a result of minimizing the denial and/or underpayment of such claims.

### Generate Propensity to Deny Data Object Module

Turning now to FIG. 8, additional details are provided regarding a process flow for generating a PTD data object for an insurance claim in accordance with various embodiments of the disclosure. Accordingly, FIG. 8 provides a flow diagram showing a generate propensity to deny (PTD) data object module for performing such functionality according to various embodiments of the disclosure.

As previously noted, the generate PTD data object module is invoked in various embodiments by the evaluate claim module to generate a PTD data object for an insurance claim as described above. However, with that said, the generate PTD data object module may be invoked by a different module or as a stand-alone module in other embodiments.

As previously discussed, in various embodiments, the PTD data object includes a plurality of potential denial data objects that represent different adjustment reason codes. Accordingly, an 835 EDI transaction returned in response to an insurance claim submission (e.g., an 837 EDI transaction) may include one or more of these adjustment reason codes to explain why the claim or service line for the claim was paid differently than it was billed. However, an important characteristic of these different adjustment reason codes is that use of the different codes is highly imbalanced, resulting in historical data on the adjustment reason codes containing more samples for some of the adjustment reason codes and less samples for others. Therefore, to address this problem, the computational framework is configured in particular embodiments to use a mixed approach in which a machine learning model is used in generating some of the potential data objects found in the PTD data object and a probabilistic statistical model is used in generating the remaining potential data objects found in the PTD data object.

The reason for using this mixed appropriate in these particular embodiments is because machine learning oftentimes requires sufficient historical data to train the model. Therefore, for some adjustment reason codes that are rarely used, the historical data does not contain enough of a sample to adequately train the machine model for these adjustment reason codes. Thus, for those adjustment reason codes that do not have enough historical data (e.g., codes with less than 1000 samples) and/or do not have suitable features for machine learning, a conditional statistical model is used in various embodiments. Specifically, in particular embodiments, the statistical model is configured to analyze the dollar amount values that payers deny paying due to one of the adjustment reason codes. The model calculates the scores that determine the most expensive and significant adjustment reason codes that should be avoided. Accordingly, in some embodiments, models may be developed for the different payers in that the scores generated can be unique for each payer. In addition, in some embodiments, a statistical model may be used for those adjustment reason codes that do not show robust results (e.g., F1 scores) during machine learning training.

In various embodiments, the development of the statistical model(s) may involve dividing the historical data by payers so that a model may be developed for each payer. Here, in particular embodiments, one or more denial quotients are calculated for each adjustment reason code using, for example, the equation:

$${Q}_{a r c}^{p a y e r} = 100 \% * \frac{Σ {M}_{a r c}^{p a y e r}}{Σ {M}^{payer}} ,$$

where ΣM^{payer} is a total adjusted monetary amount over historical period for a given payer. The ΣM_{arc}^{payer} is a denied monetary amount for a specified adjustment reason code. Q_{arc}^{payer} is a denial quotient, which is an indicator that shows how large a denied monetary amount for a specified adjustment reason relative to the total adjusted monetary amount for the payer. The result of the division is multiplied by 100% to make it similar to the PTD score, which in various embodiments is generated in a range from 0 to 100.

In the event of a new payer or a payer for which there is not enough historical data to calculate payer-related denial quotients, denial quotients for all given historical data can be calculated using the equation:

$${Q}_{a r c} = 100 \% * \frac{Σ {M}_{a r c}}{Σ m} .$$

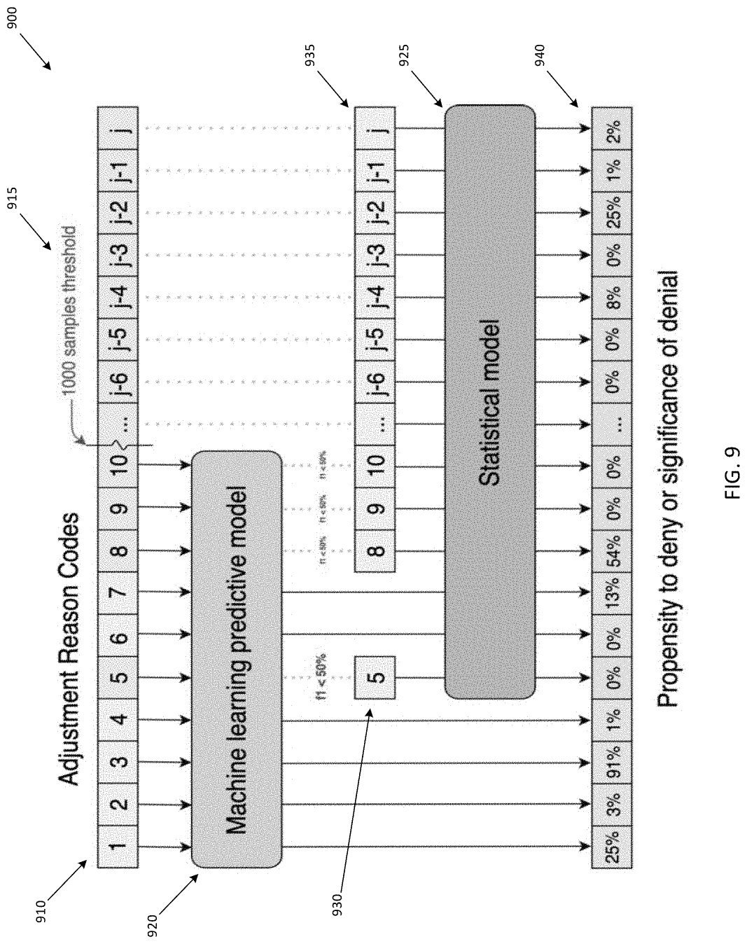

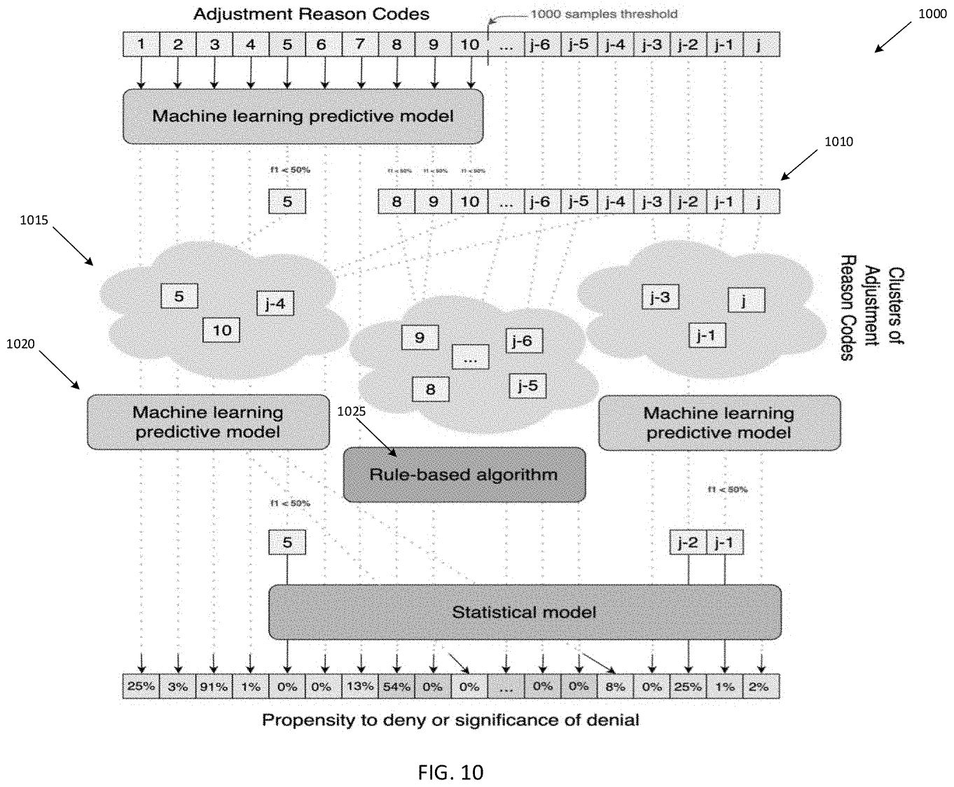

Turning briefly to FIG. 9, an example of a configuration 900 for generating a PTD data object using the machine learning model 910 and a statistical model 925 according to various embodiments is provided. Here, the machine learning model 910 is configured to provide predictions 940 for adjustment reason codes 920 that have a large enough sample size (e.g., 1000 or more samples 915) to allow for the machine learning model 910 to be trained to an acceptable level of performance. The statistical model 925 is then used for those adjustment reason codes 930 that the machine learning model 910 cannot adequately provide predictions 940 and those adjustment reason codes 935 that do not have a large enough sample size to train the machine learning model 910 to an acceptable level for providing predictions 940 for the adjustment reason codes 935. FIG. 10 provides an example of another configuration 1000 for generating a PTD data object. For this particular configuration 1000, an approach is used in which some of the adjustment reason codes 1010 (e.g., those with not a large enough sample size to adequately train the machine learning model and/or those in which the machine learning model is unable to provide predictions to an acceptable level) are split into clusters 1015 to take advantage of cross-correlations between adjustment reason codes. For example, in particular embodiments, the adjustment reason codes may be split into clusters using expert knowledge, traditional machine learning clustering approaches such as k-means, building a target labels graph and inferring community structure of the graph, using random label space partitioning such as random k-label sets, and/or the like. As a result, a machine learning model 1020 may be trained to predict one or more of the clusters, as well as a rule-based model 1025 may be developed in identifying the significance of certain clusters in contributing to a potential denial and/or underpayment of an insurance claim.

Turning back now to FIG. 8, the process flow 800 for generating a PTD data object in various embodiments begins with the generate PTD data object module extracting the data features for the insurance claim to be used as inputs to the machine learning model and/or statistical model in Operation 810. Accordingly, in particular embodiments, the generate PTD data object module performs this particular operation by invoking the feature extraction module (FIG. 3) previously described. Once the data features have been extracted, the generate PTD data object module processes one or more of the data features using the machine learning model to generate one or more of the potential denial data objects in Operation 815. Likewise, the generate PTD data object module processes one or more of the data features using the statistical model to generate one or more of the potential denial data objects in Operation 820. Finally, the generate PTD data object module generates the PTD data object using (e.g., combining) the one or more potential denial data objects in Operation 825.

As previously noted, the PTD data object in various embodiments provides a representation of the likelihood of the insurance claim being denied and/or underpaid. Here, each of the potential denial data objects may represent a potential issue, defect, deficiency, and/or the like that may be found in the insurance claim that may contribute to the denial and/or underpayment of the insurance claim. Therefore, each of the potential denial data objects provides a representation (e.g., a value, an indicator, and/or the like) of the particular potential denial data object's contribution to the denial and/or underpayment. That is to say, each of the potential denial data objects provides a representation identifying a significance of the potential issue, defect, deficiency, and/or the like associated with the potential denial data object contributing to the insurance claim being denied and/or underpaid.

### Mitigating Action Module

Turning now to FIG. 11, additional details are provided regarding a process flow for generating one or more mitigating actions to address a potential issue, defect, deficiency, and/or the like that may be found in an insurance claim in accordance with various embodiments of the disclosure. Accordingly, FIG. 11 provides a flow diagram showing a mitigating action module for performing such functionality according to various embodiments of the disclosure.

As previously noted, the mitigating action module is invoked in various embodiments by the evaluate claim module to generate one or more mitigating actions as described above. However, with that said, the mitigating action module may be invoked by a different module or as a stand-alone module in other embodiments.

The process flow 1100 begins with the mitigating action module selecting a potential denial data object that has been identified for the insurance claim in Operation 1110. As previously noted, one or more potential denial data objects may be identified for the insurance claim that represent potential issues, defects, deficiencies, and/or the like that may be found in the insurance claim that are considered to contribute to the likelihood of the insurance claim being denied and/or underpaid with respect to a certain threshold level, significance, magnitude, weight, and/or the like. Once selected, the mitigating action module applies a mitigating model in Operation 1115 to the potential denial data object (e.g., one or more data features for the potential denial data object) to generate one or more mitigating actions that can be carried out to address the potential issue, defect, deficiency, and/or the like associated with the potential denial data object.

As previously noted, in particular embodiments, the mitigating model may be a rules-based model configured to apply rules-based logic to identify the one or more mitigating actions for the potential denial data object. Accordingly, the mitigating model is configured in these embodiments to apply logic based at least in part on rules for identifying mitigating actions that can be carried out to cure and/or remedy various issues, detects, deficiencies, and/or the like that may be found in an insurance claim that can lead to the insurance claim being denied and/or underpaid. For example, the mitigating model may include logic that identifies a mitigating action as providing documentation establishing a patient as a dependent of an insured party for a potential denial data object representing an adjustment reason code associated with the reason for non-payment is the payer's records indicate the patient is not an eligible dependent. In some embodiments, the rules-based logic may be based at least in part on historical claims denial appeal cases.

At this point, the mitigating module determines whether another potential denial data object has been identified for the insurance claim in Operation 1120. If so, then the mitigating module returns to Operation 1110, selects the next potential denial data object identified for the insurance claim, and applies the mitigating model to the newly selected potential denial data object to generate one or more mitigating actions for the potential denial data object. Once the mitigating module has processed all of the potential denial data objects identified for the insurance claim, the mitigating module provides the mitigating action(s) in Operation 1125.

### Illustrative Device Architecture

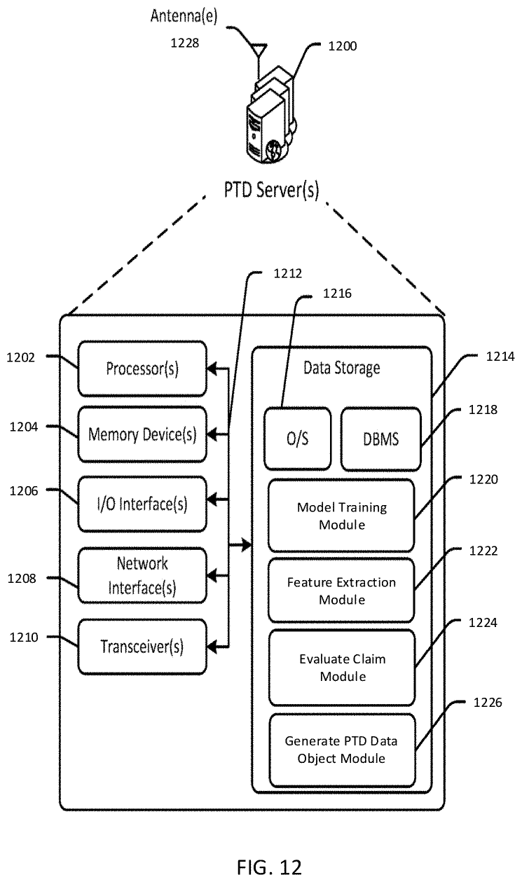

FIG. 12 is a schematic block diagram of an example propensity to deny server 1200 in accordance with one or more example embodiments of the disclosure. The propensity to deny server 1200 may include any suitable computing entity including, but not limited to, a personal computer (PC), a tablet PC, a set-top box (STB), a Personal Digital Assistant (PDA), a mobile device (smart phone), a web appliance, a server, a network router, a switch or bridge, or any other device capable of executing a set of instructions (sequential or otherwise) that specify actions to be taken by that device. Accordingly, the propensity to deny server 1200 may correspond to an illustrative device configuration for performing operations as referred to in FIGS. 1-11.

The propensity to deny server 1200 may be configured to communicate via one or more networks with one or more servers, user devices, and/or the like. Such network(s) may include, but are not limited to, any one or more different types of communications networks such as, for example, cable networks, public networks (e.g., the Internet), private networks (e.g., frame-relay networks), wireless networks, cellular networks, telephone networks (e.g., a public switched telephone network), or any other suitable private or public packet-switched or circuit-switched networks. Further, such network(s) may have any suitable communication range associated therewith and may include, for example, global networks (e.g., the Internet), metropolitan area networks (MANs), wide area networks (WANs), local area networks (LANs), or personal area networks (PANs). In addition, such network(s) may include communication links and associated networking devices (e.g., link-layer switches, routers, etc.) for transmitting network traffic over any suitable type of medium including, but not limited to, coaxial cable, twisted-pair wire (e.g., twisted-pair copper wire), optical fiber, a hybrid fiber-coaxial (HFC) medium, a microwave medium, a radio frequency communication medium, a satellite communication medium, or any combination thereof.

In an illustrative configuration, the propensity to deny server 1200 may include one or more processors (processor(s)) 1202, one or more memory devices 1204 (generically referred to herein as memory 1204), one or more input/output (“I/O”) interface(s) 1206, one or more network interface(s) 1208, one or more transceivers 1210, and data storage 1214. The propensity to deny server 1200 may further include one or more buses 1212 that functionally couple various components of the propensity to deny server 1200. The propensity to deny server 1200 may further include one or more antenna(e) 1228 that may include, without limitation, a cellular antenna for transmitting or receiving signals to/from a cellular network infrastructure, an antenna for transmitting or receiving Wi-Fi signals to/from an access point (AP), a Global Navigation Satellite System (GNSS) antenna for receiving GNSS signals from a GNSS satellite, a Bluetooth antenna for transmitting or receiving Bluetooth signals, a Near Field Communication (NFC) antenna for transmitting or receiving NFC signals, and so forth. These various components will be described in more detail hereinafter.

The data storage 1214 may include removable storage and/or non-removable storage including, but not limited to, magnetic storage, optical disk storage, and/or tape storage. The data storage 1214 may provide non-volatile storage of computer-executable instructions and other data. The memory 1204 and the data storage 1214, removable and/or non-removable, are examples of computer-readable storage media (CRSM) as that term is used herein.

The data storage 1214 may store computer-executable code, instructions, and/or the like that may be loadable into the memory 1204 and executable by the processor(s) 1202 to cause the processor(s) 1202 to perform or initiate various operations. The data storage 1214 may additionally store data that may be copied to memory 1204 for use by the processor(s) 1202 during the execution of the computer-executable instructions. Moreover, output data generated as a result of execution of the computer-executable instructions by the processor(s) 1202 may be stored initially in memory 1204 and may ultimately be copied to data storage 1214 for non-volatile storage.

More specifically, the data storage 1214 may store one or more operating systems (O/S) 1216; one or more database management systems (DBMS) 1218; and one or more program module(s), applications, engines, computer-executable code, scripts, and/or the like such as, for example, the model training module 1220, the feature extraction module 1222, the evaluate claim module 1224, and/or the generate PTD data object module 1226, as well as the transform categorical feature module, the transform service code attribute model, the transform date feature module, and the mitigating action module (not shown), as described herein. Some or all of these module(s) may be sub-module(s). Any of the components depicted as being stored in data storage 1214 may include any combination of software, firmware, and/or hardware. The software and/or firmware may include computer-executable code, instructions, and/or the like that may be loaded into the memory 1204 for execution by one or more of the processor(s) 1202. Any of the components depicted as being stored in data storage 1214 may support functionality described in reference to correspondingly named components earlier in this disclosure.

The processor(s) 1202 may be configured to access the memory 1204 and execute computer-executable instructions loaded therein. For example, the processor(s) 1202 may be configured to execute computer-executable instructions of the various program module(s), applications, engines, and/or the like of the propensity to deny server 1200 to cause or facilitate various operations to be performed in accordance with one or more embodiments of the disclosure. The processor(s) 1202 may include any suitable processing unit capable of accepting data as input, processing the input data in accordance with stored computer-executable instructions, and generating output data. The processor(s) 1202 may include any type of suitable processing unit including, but not limited to, a central processing unit, a microprocessor, a Reduced Instruction Set Computer (RISC) microprocessor, a Complex Instruction Set Computer (CISC) microprocessor, a microcontroller, an Application Specific Integrated Circuit (ASIC), a Field-Programmable Gate Array (FPGA), a System-on-a-Chip (SoC), a digital signal processor (DSP), and so forth. Further, the processor(s) 1202 may have any suitable microarchitecture design that includes any number of constituent components such as, for example, registers, multiplexers, arithmetic logic units, cache controllers for controlling read/write operations to cache memory, branch predictors, and/or the like. The microarchitecture design of the processor(s) 1202 may be capable of supporting any of a variety of instruction sets.

Referring now to functionality supported by the various program modules described herein, the model training module 1220 may include computer-executable instructions, code, and/or the like that responsive to execution by one or more of the processor(s) 1202 may perform functions and/or operations as detailed herein. The feature extraction module 1222 may include computer-executable instructions, code, and/or the like that responsive to execution by one or more of the processor(s) 1202 may perform functions and/or operations as detailed herein. The transform categorical feature module may include computer-executable instructions, code, and/or the like that responsive to execution by one or more of the processor(s) 1202 may perform functions and/or operations as detailed herein. The transform service code attribute module may include computer-executable instructions, code, and/or the like that responsive to execution by one or more of the processor(s) 1202 may perform functions and/or operations as detailed herein. The evaluate claim module 1224 may include computer-executable instructions, code, and/or the like that responsive to execution by one or more of the processor(s) 1202 may perform functions and/or operations as detailed herein. The generate PTD data object module 1226 may include computer-executable instructions, code, and/or the like that responsive to execution by one or more of the processor(s) 1202 may perform functions and/or operations as detailed herein. The mitigating action module may include computer-executable instructions, code, and/or the like that responsive to execution by one or more of the processor(s) 1202 may perform functions and/or operations as detailed herein.

Referring now to other illustrative components depicted as being stored in the data storage 1214, the O/S 1216 may be loaded from the data storage 1214 into the memory 1204 and may provide an interface between other application software executing on the propensity to deny server 1200 and hardware resources of the propensity to deny server 1200. More specifically, the O/S 1216 may include a set of computer-executable instructions for managing hardware resources of the propensity to deny server 1200 and for providing common services to other application programs (e.g., managing memory allocation among various application programs). In certain example embodiments, the O/S 1216 may control execution of the other program module(s) to dynamically enhance characters for content rendering. The O/S 1216 may include any operating system now known or which may be developed in the future including, but not limited to, any server operating system, any mainframe operating system, or any other proprietary or non-proprietary operating system.

The DBMS 1218 may be loaded into the memory 1204 and may support functionality for accessing, retrieving, storing, and/or manipulating data stored in the memory 1204 and/or data stored in the data storage 1214. The DBMS 1218 may use any of a variety of database models (e.g., relational model, object model, etc.) and may support any of a variety of query languages. The DBMS 1218 may access data represented in one or more data schemas and stored in any suitable data repository including, but not limited to, databases (e.g., relational, object-oriented, etc.), file systems, flat files, distributed datastores in which data is stored on more than one node of a computer network, peer-to-peer network datastores, and/or the like. In those example embodiments in which the propensity to deny server 1200 is a mobile device, the DBMS 1218 may be any suitable light-weight DBMS optimized for performance on a mobile device.

It should be appreciated that the program module(s), applications, computer-executable instructions, code, and/or the like described herein are merely illustrative and not exhaustive and that processing described as being supported by any particular module may alternatively be distributed across multiple module(s) or performed by a different module. In addition, various program module(s), script(s), plug-in(s), Application Programming Interface(s) (API(s)), or any other suitable computer-executable code hosted locally on the propensity to deny server 1200, and/or hosted on other computing device(s) accessible via one or more networks, may be provided to support functionality provided by the program module(s), applications, or computer-executable code described herein and/or additional or alternate functionality. Further, functionality may be modularized differently such that processing described as being supported collectively by the collection of program module(s) described herein may be performed by a fewer or greater number of module(s), or functionality described as being supported by any particular module may be supported, at least in part, by another module. In addition, program module(s) that support the functionality described herein may form part of one or more applications executable across any number of systems or devices in accordance with any suitable computing model such as, for example, a client-server model, a peer-to-peer model, and so forth. In addition, any of the functionality described as being supported by any of the program module(s) described herein may be implemented, at least partially, in hardware and/or firmware across any number of devices.

It should further be appreciated that the propensity to deny server 1200 may include alternate and/or additional hardware, software, or firmware components beyond those described or depicted without departing from the scope of the disclosure. More particularly, it should be appreciated that software, firmware, or hardware components depicted as forming part of the propensity to deny server 1200 are merely illustrative and that some components may not be present or additional components may be provided in various embodiments. While various illustrative program module(s) have been depicted and described as software module(s) stored in data storage 1214, it should be appreciated that functionality described as being supported by the program module(s) may be enabled by any combination of hardware, software, and/or firmware. It should further be appreciated that each of the above-mentioned module(s) may, in various embodiments, represent a logical partitioning of supported functionality. This logical partitioning is depicted for ease of explanation of the functionality and may not be representative of the structure of software, hardware, and/or firmware for implementing the functionality. Accordingly, it should be appreciated that functionality described as being provided by a particular module may, in various embodiments, be provided at least in part by one or more other module(s). Further, one or more depicted module(s) may not be present in certain embodiments, while in other embodiments, additional module(s) not depicted may be present and may support at least a portion of the described functionality and/or additional functionality. Moreover, while certain module(s) may be depicted and described as sub-module(s) of another module, in certain embodiments, such module(s) may be provided as independent module(s) or as sub-module(s) of other module(s).

One or more operations of the methods, process flows, and use cases of FIGS. 1-12 may be performed by a device having the illustrative configuration depicted in FIG. 12, or more specifically, by one or more engines, program module(s), applications, and/or the like executable on such a device. It should be appreciated, however, that such operations may be implemented in connection with numerous other device configurations.

The operations described and depicted in the illustrative methods and process flows of FIGS. 1-12 may be carried out or performed in any suitable order as desired in various example embodiments of the disclosure. Additionally, in certain example embodiments, at least a portion of the operations may be carried out in parallel. Furthermore, in certain example embodiments, less, more, or different operations than those depicted in FIGS. 1-12 may be performed.

Although specific embodiments of the disclosure have been described, one of ordinary skill in the art will recognize that numerous other modifications and alternative embodiments are within the scope of the disclosure. For example, any of the functionality and/or processing capabilities described with respect to a particular device or component may be performed by any other device or component. Further, while various illustrative implementations and architectures have been described in accordance with embodiments of the disclosure, one of ordinary skill in the art will appreciate that numerous other modifications to the illustrative implementations and architectures described herein are also within the scope of this disclosure.

Certain aspects of the disclosure are described above with reference to block and flow diagrams of systems, methods, apparatuses, and/or computer program products according to example embodiments. It will be understood that one or more blocks of the block diagrams and flow diagrams, and combinations of blocks in the block diagrams and the flow diagrams, respectively, may be implemented by execution of computer-executable program instructions. Likewise, some blocks of the block diagrams and flow diagrams may not necessarily need to be performed in the order presented or may not necessarily need to be performed at all, according to some embodiments. Further, additional components and/or operations beyond those depicted in blocks of the block and/or flow diagrams may be present in certain embodiments.

Accordingly, blocks of the block diagrams and flow diagrams support combinations of means for performing the specified functions, combinations of elements or steps for performing the specified functions, and program instruction means for performing the specified functions. It will also be understood that each block of the block diagrams and flow diagrams, and combinations of blocks in the block diagrams and flow diagrams, may be implemented by special-purpose, hardware-based computer systems that perform the specified functions, elements or steps, or combinations of special-purpose hardware and computer instructions.

Program module(s), applications, and/or the like disclosed herein may include one or more software components including, for example, software objects, methods, data structures, and/or the like. Each such software component may include computer-executable instructions that, responsive to execution, cause at least a portion of the functionality described herein (e.g., one or more operations of the illustrative methods described herein) to be performed.

A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform.

Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, or a report writing language. In one or more example embodiments, a software component comprising instructions in one of the foregoing examples of programming languages may be executed directly by an operating system or other software component without having to be first transformed into another form.

A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

Software components may invoke or be invoked by other software components through any of a wide variety of mechanisms. Invoked or invoking software components may comprise other custom-developed application software, operating system functionality (e.g., device drivers, data storage (e.g., file management) routines, other common routines and services, etc.), or third-party software components (e.g., middleware, encryption, or other security software, database management software, file transfer or other network communication software, mathematical or statistical software, image processing software, and format translation software).

Software components associated with a particular solution or system may reside and be executed on a single platform or may be distributed across multiple platforms. The multiple platforms may be associated with more than one hardware vendor, underlying chip technology, or operating system. Furthermore, software components associated with a particular solution or system may be initially written in one or more programming languages but may invoke software components written in another programming language.

Computer-executable program instructions may be loaded onto a special-purpose computer or other particular machine, a processor, or other programmable data processing apparatus to produce a particular machine, such that execution of the instructions on the computer, processor, or other programmable data processing apparatus causes one or more functions or operations specified in the flow diagrams to be performed. These computer program instructions may also be stored in a computer-readable storage medium (CRSM) that upon execution may direct a computer or other programmable data processing apparatus to function in a particular manner, such that the instructions stored in the computer-readable storage medium produce an article of manufacture including instruction means that implement one or more functions or operations specified in the flow diagrams. The computer program instructions may also be loaded onto a computer or other programmable data processing apparatus to cause a series of operational elements or steps to be performed on the computer or other programmable apparatus to produce a computer-implemented process.

Additional types of CRSM that may be present in any of the devices described herein may include, but are not limited to, programmable random access memory (PRAM), SRAM, DRAM, RAM, ROM, electrically erasable programmable read-only memory (EEPROM), flash memory or other memory technology, compact disc read-only memory (CD-ROM), digital versatile disc (DVD) or other optical storage, magnetic cassettes, magnetic tape, magnetic disk storage or other magnetic storage devices, or any other medium which can be used to store the information and which can be accessed. Combinations of any of the above are also included within the scope of CRSM. Alternatively, computer-readable communication media (CRCM) may include computer-readable instructions, program module(s), or other data transmitted within a data signal, such as a carrier wave, or other transmission. However, as used herein, CRSM does not include CRCM.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

### CONCLUSION

While this specification contains many specific embodiment details, these should not be construed as limitations on the scope of any invention or of what may be claimed, but rather as descriptions of features that may be specific to particular embodiments of particular inventions. Certain features that are described in this specification in the context of separate embodiments may also be implemented in combination in a single embodiment. Conversely, various features that are described in the context of a single embodiment may also be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are described in a particular order, this should not be understood as requiring that such operations be performed in the particular order described or in sequential order, or that all described operations be performed, to achieve desirable results. In certain circumstances, multitasking and parallel processing may be advantageous. Moreover, the separation of various components in the embodiments described above should not be understood as requiring such separation in all embodiments, and it should be understood that the described program components (e.g., modules) and systems may generally be integrated together in a single software product or packaged into multiple software products.

Many modifications and other embodiments of the disclosure will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for the purposes of limitation.

What is claimed is:

1. A computer-implemented method for evaluating an insurance claim prior to submission, the computer-implemented method comprising:
extracting, via one or more computer processors, a plurality of data features from data for the insurance claim;
processing, via the one or more computer processors, one or more of the plurality of data features using a machine learning model to generate an initial propensity to deny data object comprising a plurality of potential denial data objects, wherein (i) the machine learning model is configured as an ensemble of classifiers in which each classifier in the ensemble corresponds to a potential issue of a plurality of potential issues for the insurance claim and is trained to generate a data value for the potential issue representing an impact of the potential issue on the insurance claim being at least one of denied or underpaid, (ii) the initial propensity to deny data object represents a likelihood of the insurance claim being at least one of denied or underpaid, and (iii) each potential denial data object of the plurality of potential denial data objects represents a potential issue of the plurality of potential issues and comprises the corresponding data value;
processing, via the one or more computer processors, at least one potential denial data object of the plurality of potential denial data objects using a mitigating model to identify at least one mitigating action configured to cure the potential issue associated with the at least one potential denial data object;
providing, via the one or more computer processors, the potential issue associated with the at least one potential denial data object, the data value corresponding to the at least one potential denial data object, and the at least one mitigating action for display via a user interface through a user device;
receiving, via the one or more computer processors, an indication of a completion of the at least one mitigating action to cure the potential issue for the at least one potential denial data object; and
responsive to receiving the indication of the completion of the at least one mitigating action:
extracting, via the one or more computer processors, the plurality of data features from the data for the insurance claim, wherein the plurality of data features reflects the completion of the at least one mitigating action;
processing, via the one or more computer processors, one or more of the plurality of data features using the machine learning model to generate a working propensity to deny data object comprising the plurality of potential denial data objects, wherein the working propensity to deny data object represents a likelihood of the insurance claim being at least one of denied or underpaid in light of the completion of the at least one mitigating action and the at least one potential denial data object of the plurality of potential denial data objects comprises an updated data value based at least in part on the completion of the at least one mitigating action; and
providing, via the one or more computer processors, the potential issue associated with the at least one potential denial data object, the updated data value corresponding to the at least one potential denial data object, and an indication that the at least one mitigating action has been completed for display via the user interface through the user device.

2. The computer-implemented method of claim 1 further comprising:
processing, via the one or more computer processors, one or more of the plurality of data features using a statistical model to generate one or more additional potential denial data objects for the initial propensity to deny data object, wherein each additional potential denial data object of the one or more additional potential denial data objects represents an additional potential issue for the insurance claim and comprises a value representing an impact of the additional potential issue on the insurance claim being at least one of denied or underpaid.

3. The computer-implemented method of claim 2, wherein the statistical model is configured to calculate the value for an additional potential denial data object of the one or more additional potential denial data objects as a quotient comprising a denied monetary amount for the additional potential denial data object divided by a total adjusted monetary amount over a historical period for a given payer.

4. The computer-implemented method of claim 1 further comprising:
determining, via the one or more computer processors, whether the working propensity to deny data object satisfies a threshold; and
responsive to the working propensity to deny data object satisfying the threshold, automatically submitting, via the one or more computer processors, the insurance claim for payment.

5. (canceled)

6. (canceled)

7. The computer-implemented method of claim 1, wherein the user interface is embedded as one or more web service calls in at least one of a patient registration interface, a case management interface, or an electronic medical record user interface.

8. A system for evaluating an insurance claim prior to submission, the system comprising:
one or more computer processors; and
computer memory storing computer-executable instructions that are configured to, when executed by the one or more computer processors, cause the system to:
extract a plurality of data features from data for the insurance claim;
process one or more of the plurality of data features using a machine learning model to generate an initial propensity to deny data object comprising a plurality of potential denial data objects, wherein (i) the machine learning model is configured as an ensemble of classifiers in which each classifier in the ensemble corresponds to a potential issue of a plurality of potential issues for the insurance claim and is trained to generate a data value for the potential issue representing an impact of the potential issue on the insurance claim being at least one of denied or underpaid, (ii) the initial propensity to deny data object represents a likelihood of the insurance claim being at least one of denied or underpaid, and (iii) each potential denial data object of the plurality of potential denial data objects represents a potential issue of the plurality of potential issues and comprises the corresponding data value;
process at least one potential denial data object of the plurality of potential denial data objects using a mitigating model to identify at least one mitigating action configured to cure the potential issue associated with the at least one potential denial data object;
provide the potential issue associated with the at least one potential denial data object, the data value corresponding to the at least one potential denial data object, and the at least one mitigating action for display via a user interface through a user device;
receive an indication of a completion of the at least one mitigating action to cure the potential issue for the at least one potential denial data object; and
responsive to receiving the indication of the completion of the at least one mitigating action:
extract the plurality of data features from the data for the insurance claim, wherein the plurality of data features reflects the completion of the at least one mitigating action;
process one or more of the plurality of data features using the machine learning model to generate a working propensity to deny data object comprising the plurality of potential denial data objects, wherein the working propensity to deny data object represents a likelihood of the insurance claim being at least one of denied or underpaid in light of the completion of the at least one mitigating action and the at least one potential denial data object of the plurality of potential denial data objects comprises an updated data value based at least in part on the completion of the at least one mitigating action; and
provide the potential issue associated with the at least one potential denial data object, the updated data value corresponding to the at least one potential denial data object, and an indication that the at least one mitigating action has been completed for display via the user interface through the user device.

9. The system of claim 8, wherein the computer-executable instructions that are configured to, when executed by the one or more computer processors, cause the system to:
process one or more of the plurality of data features using a statistical model to generate one or more additional potential denial data objects for the initial propensity to deny data object, wherein each additional potential denial data object of the one or more additional potential denial data objects represents an additional potential issue for the insurance claim and comprises a value representing an impact of the additional potential issue on the insurance claim being at least one of denied or underpaid.

10. The system of claim 9, wherein the statistical model is configured to calculate the value for an additional potential denial data object of the one or more additional potential denial data objects as a quotient comprising a denied monetary amount for the additional potential denial data object divided by a total adjusted monetary amount over a historical period for a given payer.

11. The system of claim 8, wherein the computer-executable instructions that are configured to, when executed by the one or more computer processors, cause the system to:
determine whether the working propensity to deny data object satisfies a threshold; and
responsive to the working propensity to deny data object satisfying the threshold, automatically submit the insurance claim for payment.

12. (canceled)

13. (canceled)

14. The system of claim 8, wherein the user interface is embedded as one or more web service calls in at least one of a patient registration interface, a case management interface, or an electronic medical record user interface.

15. A non-transitory computer-readable medium storing computer-executable instructions for evaluating an insurance claim prior to submission, the computer-executable instructions are configured to, when executed by one or more computer processors, cause the one or more computer processors to:
extract a plurality of data features from data for the insurance claim;
process one or more of the plurality of data features using a machine learning model to generate an initial propensity to deny data object comprising a plurality of potential denial data objects, wherein (i) the machine learning model is configured as an ensemble of classifiers in which each classifier in the ensemble corresponds to a potential issue of a plurality of potential issues for the insurance claim and is trained to generate a data value for the potential issue representing an impact of the potential issue on the insurance claim being at least one of denied or underpaid, (ii) the initial propensity to deny data object represents a likelihood of the insurance claim being at least one of denied or underpaid, and (iii) each potential denial data object of the plurality of potential denial data objects represents a potential issue of the plurality of potential issues and comprises the corresponding data value;
process at least one potential denial data object of the plurality of potential denial data objects using a mitigating model to identify at least one mitigating action configured to cure the potential issue associated with the at least one potential denial data object;
provide the potential issue associated with the at least one potential denial data object, the data value corresponding to the at least one potential denial data object, and the at least one mitigating action for display via a user interface through a user device;
receive an indication of a completion of the at least one mitigating action to cure the potential issue for the at least one potential denial data object; and
responsive to receiving the indication of the completion of the at least one mitigating action:
extract the plurality of data features from the data for the insurance claim, wherein the plurality of data features reflects the completion of the at least one mitigating action;
process one or more of the plurality of data features using the machine learning model to generate a working propensity to deny data object comprising the plurality of potential denial data objects, wherein the working propensity to deny data object represents a likelihood of the insurance claim being at least one of denied or underpaid in light of the completion of the at least one mitigating action and the at least one potential denial data object of the plurality of potential denial data objects comprises an updated data value based at least in part on the completion of the at least one mitigating action; and
provide the potential issue associated with the at least one potential denial data object, the updated data value corresponding to the at least one potential denial data object, and an indication that the at least one mitigating action has been completed for display via the user interface through the user device.

16. The non-transitory computer-readable medium of claim 15, wherein the computer-executable instructions are configured to, when executed by the one or more computer processors, cause the one or more computer processors to:
process one or more of the plurality of data features using a statistical model to generate one or more additional potential denial data objects for the initial propensity to deny data object, wherein each additional potential denial data object of the one or more additional potential denial data objects represents an additional potential issue for the insurance claim and comprises a value representing an impact of the additional potential issue on the insurance claim being at least one of denied or underpaid.

17. The non-transitory computer-readable medium of claim 16, wherein the statistical model is configured to calculate the value for an additional potential denial data object of the one or more additional potential denial data objects as a quotient comprising a denied monetary amount for the additional potential denial data object divided by a total adjusted monetary amount over a historical period for a given payer.

18. The non-transitory computer-readable medium of claim 15, wherein the computer-executable instructions are configured to, when executed by the one or more computer processors, cause the one or more computer processors to:
determine whether the working propensity to deny data object satisfies a threshold; and
responsive to the working propensity to deny data object satisfying the threshold, automatically submit the insurance claim for payment.

19. (canceled)

20. (canceled)

21. The non-transitory computer-readable medium of claim 15, wherein the user interface is embedded as one or more web service calls in at least one of a patient registration interface, a case management interface, or an electronic medical record user interface.

22. The method of claim 1, wherein the plurality of data features comprises data provided in an 837 electronic data interchange transaction.

23. The method of claim 1, wherein the plurality of potential denial data objects comprises a plurality of adjustment reason codes provided in an 835 electronic data interchange transaction.

24. The system of claim 8, wherein the plurality of data features comprises data provided in an 837 electronic data interchange transaction.

25. The system of claim 8, wherein the plurality of potential denial data objects comprises a plurality of adjustment reason codes provided in an 835 electronic data interchange transaction.

26. The non-transitory computer-readable medium of claim 15, wherein the plurality of data features comprises data provided in an 837 electronic data interchange transaction.

27. The non-transitory computer-readable medium of claim 15, wherein the plurality of potential denial data objects comprises a plurality of adjustment reason codes provided in an 835 electronic data interchange transaction.
